(19) United States
(12) Patent Application Publication
(10) Pub. No.: US 2005/0214749 A1
(43) Pub. Date: Sep. 29, 2005

(54) Method for determining reduced susceptibility of HIV to protease inhibitor treatment

(76) Inventors: Colombe Chappey, San Francisco, CA (US); Christos J. Petropoulos, Half Moon Bay, CA (US); Neil T. Parkin, Belmont, CA (US)

Correspondence Address: JONES DAY, 222 EAST 41ST ST, NEW YORK, NY, 10017, US

(21) Appl. No.: 11/051,812

(22) Filed: Feb. 4, 2005

Related U.S. Application Data
(60) Provisional application No. 60/542,795, filed on Feb. 6, 2004.

(51) Int. Cl. C12Q 1/70; C12Q 1/68

(52) U.S. Cl. 435/5; 435/6

(57) ABSTRACT

The present invention provides methods and devices for predicting whether a HIV variant will be resistant to an antiviral drug based on the variant's genotype. In one aspect, methods are provided comprising determining whether a combination of protease inhibitor resistance mutations meet certain conditions, as disclosed herein, thereby assessing the effectiveness of ritonavir-boosted indinavir therapy in the HIV-infected subject. Computer implemented methods comprising determining HIV resistance are provided.

This application is entitled to and claims benefit of U.S. Provisional Application No. 60/542,795, filed Feb. 6, 2004, which is hereby incorporated by reference in its entirety.

### 1. FIELD OF THE INVENTION

This invention relates to methods and devices for determining the susceptibility of a pathogenic virus to an anti-viral compound. In particular, this invention relates to methods and devices useful for the identification of HIV resistance to ritonavir-boosted indinavir therapy in a subject infected with HIV using genotypic information of the HIV.

### 2. BACKGROUND OF THE INVENTION

More than 60 million people have been infected with the human immunodeficiency virus (“HIV”), the causative agent of acquired immune deficiency syndrome (“AIDS”), since the early 1980s. See Lucas, 2002, Lepr Rev. 73(1):64-71. HIV/AIDS is now the leading cause of death in sub-Saharan Africa, and is the fourth biggest killer worldwide. At the end of 2001, an estimated 40 million people were living with HIV globally. See Norris, 2002, Radiol Technol. 73(4):339-363.

The goal of antiretroviral therapy drug treatment is to delay disease progression and prolong survival by achieving sustained suppression of viral replication. Current anti-HIV drugs target different stages of the HIV life cycle and a variety of enzymes essential for HIV's replication and/or survival. For example, certain drugs approved for AIDS therapy inhibit HIV replication by interfering with the enzymatic activities of either protease (“PR”) or reverse transcriptase (“RT”). Amongst the approved drugs are nucleoside reverse transcriptase inhibitors such as AZT, ddI, ddC, d4T, 3TC, abacavir, nucleotide reverse transcriptase inhibitors such as tenofovir, non-nucleoside reverse transcriptase inhibitors such as nevirapine, efavirenz, delavirdine and protease inhibitors (“PRIs”) such as saquinavir, ritonavir, indinavir, nelfinavir, amprenavir and lopinavir.

One consequence of the action of an anti-viral drug is that it can exert sufficient selective pressure on virus replication to select for drug-resistant mutants. Herrmann et al., 1977, Ann NY Acad Sci 284:632-637. With increasing drug exposure, the selective pressure on the replicating virus population increases to promote the more rapid emergence of drug resistant mutants.

With the inevitable emergence of drug resistance, strategies must be designed to optimize treatment in the face of resistant virus populations. Ascertaining the contribution of drug resistance to drug failure is difficult because patients that are likely to develop drug resistance are also likely to have other factors that predispose them to a poor prognosis. Richman, 1994, AIDS Res Hum Retroviruses 10:901-905. In addition, each patient typically harbors a diverse mixture of mutant strains of the virus with different mutant strains having different susceptibilities to anti-viral drugs.

Antiviral drug susceptibility assays for clinical HIV isolates are required to monitor the development of drug resistance during therapy. Ideally, assays that determine the drug susceptibility of HIV isolates should be rapid, reproducible, non-hazardous, applicable to all samples, and cost-effective. Two general approaches are now used for measuring resistance to anti-viral drugs. The first approach, called phenotypic testing, measures the susceptibility of virus taken from an infected person's virus to particular anti-viral drugs in an in vitro assay system. See, e.g., Kellam & Larder, 1994, Antimicrobial Agents and Chemo. 38:23-30; Petropoulos et al., 2000, Antimicrob. Agents Chemother. 44:920-928; Hertogs et al., 1998, Antimicrob Agents Chemother 42(2):269-76. The second approach, genotypic testing, involves identifying the presence of mutations in the HIV nucleic acid that confer resistance to certain antiviral drugs in a patient infected with that virus.

Genotypic testing, in some aspects, promises certain advantages over phenotypic testing since the facilities necessary for genotypic testing are generally cheaper and less complex than those for phenotypic testing, and genotyping is typically less labor intensive to perform and results can be had in less time. However, in order to deduce the viral sensitivity from a given genotype, the effect on drug resistance of particular resistance mutations need to be known. An additional complication of gentoypic assays is that the manual interpretation of such assays is difficult because a large number of drug resistance mutations interact in complex patterns.

Therefore, need exists not only for assessing the pertinent set of mutations relevant to a given antiviral drug therapy, but methods and devices that apply rules assigning a level of resistance to a drug or drug combination on the basis of a pattern of mutations. For example, previous studies have identified the clinically relevant susceptibility threshold for reduced susceptibility to ritonavir (“RTV”)-boosted indinavir (“IDV”) (IDV/RTV 800 mg/200 mg b.i.d.) using the PHENOSENSE™ phenotypic assay. Szumiloski et al., 2002, Antivir Ther 7:S127,2002). However, no robust genotypic correlates of reduced susceptibility to IDV/RTV therapy have been defined. As such, no genotypic assay is presently available for assessing the efficacy of IDV/RTV treatment in an HIV-infected patient.

### 3. SUMMARY OF THE INVENTION

In one aspect, the present invention provides a method of determining whether a likelihood exists for reduced protease inhibitor (“PRI”) susceptibility of a HIV population in a subject comprising identifying whether nucleic acid obtained from HIV of the subject contains one or more primary mutations in the nucleic acid encoding codon 46, 48, 82, 84, or 90 of HIV protease, identifying whether one or more secondary mutations are present in the nucleic acid encoding codon 10, 20, 24, 32, 33, 34, 36, 43, 46, 47, 48, 54, 63, 71, 73, 82, 84, 88, 89, or 90 of HIV protease, and determining whether a condition is met wherein the presence of one primary mutation and at least six secondary mutations are identified, or the presence of two primary mutations and at least four secondary mutations are identified, or three or more primary mutations and at least one secondary mutation are identified, with the proviso that an identified primary mutation may not also be counted as a secondary mutation, such that if it is determined that one of the conditions is met then the likelihood for reduced PRI susceptibility of the HIV exists.

In one embodiment of the method described above, the primary mutation encodes an amino acid in the HIV protease selected from the group consisting of M46I/L/V, G48M/S/V, V82A/F/S/T, I84A/V, and L90M and the secondary mutation encodes an amino acid in the HIV protease selected from the group consisting of L10I/F/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M.

In another embodiment, the PRI is IDV/RTV.

In an embodiment, the HIV of the subject determined to have a likelihood for reduced PRI susceptibility exhibits a 10-fold change in a PHENOSENSE™ phentotypic HIV assay compared to a reference HIV.

In one embodiment, the reference HIV is the NL4-3 strain of HIV.

In another embodiment, the PRI is IDV, the primary mutation encodes an amino acid in the HIV protease selected from the group consisting of M46I/L/V, G48M/S/V, V82A/F/S/T, I84A/V, and L90M, and the secondary mutation encodes an amino acid in the HIV protease selected from the group consisting of L10I/F/R/V, K20I/M/R/T, L24I, V32I, L33F, M36I/L, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, and L90M.

In another aspect, the present invention provides a method for assessing the effectiveness of IDV/RTV therapy in a HIV-infected subject comprising determining whether a nucleic acid obtained from HIV of the subject contains one or more primary mutations where the one or more primary mutations are in the nucleic acid encoding codon 46, 48, 82, 84, or 90 of HIV protease, and one or more secondary mutations where the one or more secondary mutations are in the nucleic acid encoding codon 10, 20, 24, 32, 33, 34, 36, 43, 46, 47, 48, 54, 63, 71, 73, 82, 84, 88, 89, or 90 of HIV protease, in a combination of one primary mutation and at least six secondary mutations, or two primary mutations and at least four secondary mutations, or three or more primary mutations and at least one secondary mutation, wherein a mutation counted as a primary mutation may not also be counted as a secondary mutation, such that the presence of such a combination indicates a decrease in susceptibility to IDV/RTV, thereby assessing the effectiveness of IDV/RTV therapy in the subject.

In one embodiment, the primary mutations encode for an amino acid selected from the group consisting of M46I/L/V, G48M/S/V, V82A/F/S/T, I84A/V, and L90M, and the secondary mutations encode for an amino acid selected from the group consisting of L10I/F/R/V, K20I/M/R/T, L24I, V321, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M.

In another embodiment, a decrease in susceptibility to IDV/RTV therapy is equal to or greater than a clinical cutoff value of 10-fold.

In one aspect, the present invention provides a computer implemented method of identifying a HIV population as being less susceptible to IDV/RTV in a subject infected with the HIV population, comprising inputting to a computer system data representing the genotype of a nucleic acid encoding HIV protease obtained from HIV of the subject; performing a first comparison of the genotype of the nucleic acid encoding codons 46, 48, 82, 84, or 90 of HIV protease to a database in the computer wherein the database includes nucleic acid genotypes encoding mutant codons L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M, such that if a match is identified in the first comparison, a second comparison of the genotype of the nucleic acid encoding codons 10, 20, 24, 32, 33, 34, 36, 43, 46, 47, 48, 54, 63, 71, 73, 82, 84, 88, 89, or 90 of HIV protease to the database is performed; and determining whether a condition is met that one match is made in the first comparison and at least six matches are made in the second comparison, or two matches are made in the first comparison and at least four matches are made in the second comparison, or three or more matches are made in the first comparison and at least one match is made in the second comparison, with the proviso that a match made in the first comparison may not be counted as a match in the second comparison, such that the HIV population is identified as being less susceptible to IDV/RTV therapy in a subject infected with the HIV population if it is determined that a condition is met.

In one embodiment, the computer implemented method further comprises displaying a result indicating whether or not that the HIV population is identified as being less susceptible to IDV/RTV in a subject infected with the HIV. For example, the result may be displayed on a tangible medium such as paper or other form of printout or on a computer screen, or other tangible media without limitation.

In another embodiment of the computer implemented method described above, the inputted data have been converted from a hybridization pattern of the HIV nucleic acid onto an oligonucleotide probe array attached to a solid phase.

Another aspect of the present invention provides an article of manufacture that comprises computer-readable instructions for performing the computer implemented methods of the invention. For example, the article of manufacture can be a floppy disk, CD, DVD, magnetic tape, and so forth, without limitation.

In another aspect, the present invention provides a computer system that is configured to perform the computer implemented methods of the invention.

In another aspect, the present invention provides a computer program product that identifies a subject infected with HIV as being resistant to IDV/RTV drug treatment, comprising a computer code that receives input corresponding to the genotype of the HIV nucleic acid encoding HIV protease obtained from the subject; a computer code that performs a first comparison to determine if an amino acid encoded by HIV protease codons 46, 48, 82, 84 and 90 of the HIV nucleic acid matches one or more of mutant amino acids M46I/L/V, G48M/S/V, V82A/F/S/T, I84A/V, and L90M of HIV protease; a computer code that performs a second comparison to determine if an amino acid encoded by HIV protease codons 10, 20, 24, 32, 33, 34, 36, 43, 46, 47, 48, 54, 63, 71, 73, 82, 84, 88, 89 and 90 of the HIV nucleic acid matches one or more of mutant amino acids L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/N/C, A71I/L/V/T, G73A/C/S/T, V82A/F/S/T, I84A/V, N88S/T, L89V, and L90M; a computer code that determines whether a condition is met that one match is made in the first comparison and at least six matches are made in the second comparison, or two matches are made in the first comparison and at least four matches are made in the second comparison, or three or more matches are made in the first comparison and at least one match are made in the second comparison, with the proviso that a match made in the first comparison may not be counted as a match in the second comparison, wherein the subject is identified as being resistant to IDV/RTV drug treatment if such a condition is determined to be met; a computer code that conveys a result representing whether or not subject is identified as being resistant to IDV/RTV drug treatment to an output device; and a computer readable medium that stores the computer codes.

In one embodiment of the above computer program product, input corresponding to the genotype of the HIV nucleic acid encoding HIV protease has been obtained from a hybridization pattern of the HIV nucleic acid onto an oligonucleotide array attached to a solid phase.

In other embodiments, the output device is a printer or a computer screen.

Another aspect of the present invention is a tangible medium storing the result conveyed to the output device by the computer program product described above. In an embodiment the tangible medium is a printout. In another embodiment, the tangible medium is a CD or DVD.

Another aspect of the invention provides a system of providing information of whether a HIV-infected subject is resistant to IDV/RTV, comprising: obtaining a genotype for HIV protease obtained from the subject; identifying the presence or absence of a primary mutation in the HIV comprising M46I/L/V, G48M/S/V, V82A/F/S/T, I84A/V, or L90M of HIV protease; identifying the presence or absence of a secondary mutation in the HIV comprising L10I/F/R/V, K20I/M/R/T, L24I, V32I, L33F, E34Q, M36I/L, K43T, M46I/L/V, 147A/V, G48M/S/V, I54A/L/M/S/T/V, L63P/S/A/T/Q/V/C, A71I/L/V/T, G73A/C/S/T, V82A/F/S/T, I84A/V, N88S/T, L89V, or L90M of HIV protease, wherein the secondary mutation may not also be a primary mutation; determining whether a condition is met wherein: the presence of one primary mutation and at least six secondary mutations are identified, or the presence of two primary mutations and at least four secondary mutations are identified, or three or more primary mutations and at least one secondary mutation are identified, such that if one of the conditions is met, then the subject is resistant to IDV/RTV; preparing a tangible medium comprising an indication of whether or not the subject is resistant to IDV/RTV as determined.

In one embodiment, the system further comprises conveying the tangible medium to the subject or a health care provider.

### 4. BRIEF DESCRIPTION OF THE DRAWINGS

FIG. 1 provides a example of decision tree applicable for categorizing a HIV as being resistance or sensitive.

FIG. 2 provides a comparison of discordance levels observed when one primary mutation is required along with different numbers of secondary mutations that vary between zero to seven in order to for a HIV to be categorized as genotypically resistant (“GR”) to IDV.

FIG. 3 illustrates that the minimal percentage of discordant samples is 16.3% when the rule for categorizing samples as GR to IDV/RTV is to select for samples having one primary mutation and at least four secondary mutations.

FIG. 4 is a graph of discordance levels obtained by varying the rules for categorizing an HIV sample as GR to IDV/RTV. In this example, the minimum discordance (12%) is reached utilizing an algorithm of one primary mutation and six or more secondary mutations or two primary mutations and four or more secondary mutations.

FIG. 5 compares the discordance rates taken from FIGS. 1 and 2 and illustrates that a discordance minimum of 10.6% can be reached for detecting IDV/RTV resistance using an algorithm as described herein.

FIG. 6 illustrates how exemplary genotyping interpretations rules might be incorporated into an algorithm.

FIG. 7 represents an exemplary printout of a result using the methods of the instant invention.

### 5. DETAILED DESCRIPTION OF THE INVENTION

The present invention provides methods and devices for identifying HIV populations that are resistant to protease inhibitor using a genotype interpretation algorithm. In these methods, the genotype of a HIV is matched to a primary set and a secondary set of genotypes that correspond to optimum protease mutations, as described below, and depending on the number and type (i.e., primary or secondary) of matches dictated in the algorithm, the HIV can be categorized as being resistant or susceptible to protease inhibitor. Evidence presented herein indicate that the application of the newly created algorithm to the optimum protease mutations for IDV/RTV results in a correct call of whether a HIV meets or exceeds the relevant clinical threshold of 10 FC for IDV/RTV approximately 89 out of 100 times.

5.1 Abbreviations

“HIV” is an abbreviation for human immunodeficiency virus. “PR” is an abbreviation for protease. “PRI” is an abbreviation for protease inhibitor. “PCR” is an abbreviation for polymerase chain reaction. “IDV” is an abbreviation for the protease inhibitor indinavir. “IDV/RTV” is an abbreviation for ritonavir-boosted indinavir. “FC” is an abbreviation for fold change. “GR,” “GS,” “PR” and “PS” are abbreviations for genotypically resistant, genotypically susceptible, phenotypically resistant and phenotypically susceptible, respectively.

The amino acid notations used herein for the twenty genetically encoded L-amino acids are conventional and are as follows:

| | One-Letter | Three Letter |
|---|---|---|
| Amino Acid | Abbreviation | Abbreviation |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

Unless noted otherwise, when polypeptide sequences are presented as a series of one-letter and/or three-letter abbreviations, the sequences are presented in the N-terminus to C-terminus direction, in accordance with common practice.

Substituted or mutant amino acids in HIV protease positions are represented herein in an abbreviated fashion such as “M36I/L/V,” where “M” is single-letter representation of the non-mutant reference amino acid methionine at position “36” of HIV protease, and “I,” “L” and “V” represent single-letter representations of possible mutant amino acids that may be substituted for M at position 36 in the protease.

5.2 Terminology

As used herein, “genotypic data” are data about the genotype of, for example, a virus. Examples of genotypic data include, but are not limited to, the nucleotide or amino acid sequence of a virus, a part of a virus, a viral gene, a part of a viral gene, or the identity of one or more nucleotides or amino acid residues in a viral nucleic acid or protein.

Unless otherwise specified, “primary mutations” are those occurring at positions 46, 48, 82, 84, 90 and “secondary mutations” are those occurring at 10, 20, 24, 32, 33, 34, 36, 43, 46, 47, 48, 54, 63, 71, 73, 82, 84, 88, 89, and 90. Where a particular mutation is listed as both a primary mutation and a secondary mutation, typically in the application of a rule that mutation must be one type of mutation or the other but not be counted as both a primary and a secondary mutation. In certain embodiments, primary mutations are M46I/L/V, G48M/S/V, V82A/F/S/T, I84A/V, and L90M and secondary mutations are L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M identified as optimum sets of IDV/RTV-resistance protease mutations in the context of the instant invention, as described below.

A “reference HIV” as used herein, is a HIV known to those of skill in the art to be a well-characterized drug-sensitive virus. For example, a reference HIV is NL4-3 (GenBank accession no. AF324493, incorporated by reference in its entirety for all purposes).

“Susceptibility” refers to a virus' response to a particular drug. A virus that is less susceptible or has decreased susceptibility to a drug is less sensitive or more resistant to the drug. A virus that has increased or enhanced or greater susceptibility to a drug has an increased sensitivity or decreased resistance to the drug.

Phenotypic drug susceptibility is measured as the concentration of drug required to inhibit virus replication by 50% (IC₅₀). As used herein, a “fold change” or “FC” is the ratio of a viral variant IC₅₀ divided by the IC₅₀ of a reference HIV. An FC of 1.0 indicates that the viral vaiant exhibits the same degree of drug susceptibility as the reference virus.

For drugs where sufficient clinical outcome data have been gathered, it is possible to define a clinical threshold or cutoff value. A clinical threshold or cutoff value defines the point above which the utility of a given drug begins to decline based on virological response data from clinical trials. It represents a point of increasing resistance and decreasing sensitivity of the HIV to a particular drug. The cutoff value is different for different anti-viral agents. Clinical cutoff values are determined in clinical trials by evaluating resistance and outcome data. Drug susceptibility is measured at treatment initiation. Treatment response, such as change in viral load, is monitored at predetermined time points through the course of the treatment. The drug susceptibility is correlated with treatment response and the clinical cutoff value is determined by resistance levels associated with treatment failure (statistical analysis of overall trial results).

The clinical cutoff has been identified as a 10-fold change (“FC”) for the PHENOSENSE™ phenotypic HIV assay for ritonavir-boosted indinavir. See Parkin et al., 2004, Antimicrob. Agents Chemother., 48: 437-443, incorporated by reference in its entirety for all purposes. With respect to HIV populations identified or determined to be “less susceptible” or to be “resistant,” for example, less susceptible, or resistant, to IDV/RTV or IDV/RTV therapy in a subject, as used herein, such HIV populations generally meet or exceed a 10-fold change (“FC”).

The terms “peptide,” “polypeptide” and “protein” are used interchangeably throughout.

The terms “polynucleotide,” “oligonucleotide” and “nucleic acid” are used interchangeably throughout.

The term “concordance” as used herein, means that a genotype from an HIV sample categorized as GR or GS according to an algorithm matches the phenotype (PR or PS) of that HIV sample.

The term “discordance” as used herein, means that a genotype from an HIV sample categorized as GR or GS according to an algorithm does not match the phenotype of the that HIV sample. Discordance samples include both false negatives (GS-PR) and false positive (GR-PS) identifications.

The methods and devices of the present invention arise, in part, out of the creation of an algorithm that predicts HIV resistance to IDV/RTV based on a HIV's geneotype. The methods and devices disclosed herein significantly increase the availability of information to health care professionals and HIV infected persons for making informed choices regarding IDV/RTV drug therapy.

5.3 Identifying a Protease Inhibitor-Resistance HIV

In certain aspects of the invention, methods are provided for determining whether a likelihood exists for reduced PRI susceptibility of a HIV in a subject utilizing genotype interpretation algorithms as described herein. In certain embodiments, the method comprises identifying the absence or presence of a primary mutation in a HIV nucleic acid obtained from the subject. In certain embodiments, the HIV nucleic acid encodes HIV protease. In certain embodiments, the primary mutation is a mutation in the nucleic acid encoding codon 46, 48, 82, 84, or 90 of HIV protease. In certain embodiments, the primary mutation is a mutation in the nucleic acid encoding codon 46 of HIV protease. In certain embodiments, the primary mutation is a mutation in the nucleic acid encoding codon 48of HIV protease. In certain embodiments, the primary mutation is a mutation in the nucleic acid encoding codon 82 of HIV protease. In certain embodiments, the primary mutation is a mutation in the nucleic acid encoding codon 84of HIV protease. In certain embodiments, the primary mutation is a mutation in the nucleic acid encoding codon 90 of HIV protease. In certain embodiments, the primary mutation is selected from the group consisting of two, three, and four mutations selected from the group consisting of mutations in codons 46, 48, 82, 84, or 90 of HIV protease.

In certain embodiments, the methods further comprise identifying the absence or presence of a secondary mutation in the HIV nucleic acid. In certain embodiments, the secondary mutation is a mutation in the nucleic acid encoding codon 10, 20, 24, 32, 33, 34, 36, 43, 46, 47, 48, 54, 63, 71, 73, 82, 84, 88, 89, or 90 of HIV protease. In certain embodiments, the secondary mutation is a mutation in the nucleic acid encoding codon 10 of HIV protease. In certain embodiments, the secondary mutation is a mutation in the nucleic acid encoding codon 20 of HIV protease. In certain embodiments, the secondary mutation is a mutation in the nucleic acid encoding codon 24 of HIV protease. In certain embodiments, the secondary mutation is a mutation in the nucleic acid encoding codon 32 of HIV protease. In certain embodiments, the secondary mutation is a mutation in the nucleic acid encoding codon 33 of HIV protease. In certain embodiments, the secondary mutation is a mutation in the nucleic acid encoding codon 34 of HIV protease. In certain embodiments, the secondary mutation is a mutation in the nucleic acid encoding codon 36 of HIV protease. In certain embodiments, the secondary mutation is a mutation in the nucleic acid encoding codon 43 of HIV protease. In certain embodiments, the secondary mutation is a mutation in the nucleic acid encoding codon 46 of HIV protease. In certain embodiments, the secondary mutation is a mutation in the nucleic acid encoding codon 47 of HIV protease. In certain embodiments, the secondary mutation is a mutation in the nucleic acid encoding codon 48 of HIV protease. In certain embodiments, the secondary mutation is a mutation in the nucleic acid encoding codon 54 of HIV protease. In certain embodiments, the secondary mutation is a mutation in the nucleic acid encoding codon 63 of HIV protease. In certain embodiments, the secondary mutation is a mutation in the nucleic acid encoding codon 71 of HIV protease. In certain embodiments, the secondary mutation is a mutation in the nucleic acid encoding codon 73 of HIV protease. In certain embodiments, the secondary mutation is a mutation in the nucleic acid encoding codon 82 of HIV protease. In certain embodiments, the secondary mutation is a mutation in the nucleic acid encoding codon 84 of HIV protease. In certain embodiments, the secondary mutation is a mutation in the nucleic acid encoding codon 88 of HIV protease. In certain embodiments, the secondary mutation is a mutation in the nucleic acid encoding codon 89 of HIV protease. In certain embodiments, the secondary mutation is a mutation in the nucleic acid encoding codon 90 of HIV protease. In certain embodiments, the secondary mutation is selected from the group consisting of any two, three, four, five, six, seven, eight, nine, ten, eleven, twelve, thirteen, fourteen, fifteen, sixteen, seventeen, eighteen, and nineteen mutations in a codon selected from the group consisting of codon 10, 20, 24, 32, 33, 34, 36, 43, 46, 47, 48, 54, 63, 71, 73, 82, 84, 88, 89, or 90 of HIV protease.

In certain embodiments, the methods further comprise determining whether a condition is met that (i) the presence of one primary mutation and at least six secondary mutations are identified; or (ii) the presence of two primary mutations and at least four secondary mutations are identified; or (iii) three or more primary mutations and at least one secondary mutation are identified. In certain embodiments, the methods further comprise determining whether a condition is met that the presence of one primary mutation and at least six secondary mutations are identified. In certain embodiments, the methods further comprise determining whether a condition is met that the presence of two primary mutations and at least four secondary mutations are identified. In certain embodiments, the methods further comprise determining whether a condition is met that three or more primary mutations and at least one secondary mutation are identified. In certain embodiments, an identified primary mutation may also not be counted as a secondary mutation. Where a condition (i), (ii), or (ii) is met, then the likelihood for reduced PRI susceptibility of a HIV in a subject exists.

In certain embodiments, the primary mutation encodes an amino acid in the HIV protease selected from the group consisting of M46I/L/V, G48M/S/V, V82A/F/S/T, I84A/V, and L90M. In certain embodiments, the primary mutation is M46I/L/V. In certain embodiments, the primary mutation is G48M/S/V. In certain embodiments, the primary mutation is V82A/F/S/T. In certain embodiments, the primary mutation is I84A/V. In certain embodiments, the primary mutation is L90M. In certain embodiments, the primary mutation is selected from the group consisting of two, three, and four mutations selected from the group consisting of M46I/L/V, G48M/S/V, V82A/F/S/T, I84A/V, and L90M.

In certain embodiments, the secondary mutation encodes an amino acid in the HIV protease selected from the group consisting of L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M. In certain embodiments, the secondary mutation is L10I/F/R/V. In certain embodiments, the secondary mutation is K20I/M/R/T. In certain embodiments, the secondary mutation is L24I. In certain embodiments, the secondary mutation is V32I. In certain embodiments, the secondary mutation is L33F. In certain embodiments, the secondary mutation is E34Q. In certain embodiments, the secondary mutation is M36I/L. In certain embodiments, the secondary mutation is K43T. In certain embodiments, the secondary mutation is M46I/L/V. In certain embodiments, the secondary mutation is I47A/V. In certain embodiments, the secondary mutation is G48M/S/V. In certain embodiments, the secondary mutation is I54A/L/M/S/T/V. In certain embodiments, the secondary mutation is L63P/S/A/T/Q/V/C. In certain embodiments, the secondary mutation is A71I/L/V/T. In certain embodiments, the secondary mutation is G73A/C/S/T. In certain embodiments, the secondary mutation is V82A/F/S/T. In certain embodiments, the secondary mutation is I84A/V. In certain embodiments, the secondary mutation is N88S/T. In certain embodiments, the secondary mutation is L89V. In certain embodiments, the secondary mutation is L90M. In certain embodiments, the secondary mutation is selected from the group consisting of any two, three, four, five, six, seven, eight, nine, ten, eleven, twelve, thirteen, fourteen, fifteen, sixteen, seventeen, eighteen, and nineteen mutations selected from the group consisting of L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M.

In a preferred embodiment, the protease inhibitor is IDV/RTV.

In certain embodiments, the HIV in the subject is about 10 times less susceptible to IDV/RTV than that of a reference HIV. An exemplary reference HIV is NL4-3.

The algorithms utilized in the methods of the invention have been developed by analysis and evaluation of the genotypes of a large dataset HIV of known phenotypes to determine optimum sets of protease mutations and combinations of these mutations that confer resistance to protease inhibitors. The following describes generally methods of generating genotype interpretation algorithms for the purpose of identifying drug resistant viruses.

5.3.1 Correlating Phenotypic and Genotypic Resistance to Protease Inhibitors

Datasets of viral variants with identified phenotypes can be used to correlate phenotypic and genotypic resistance to PRIs.

Generally, a phenotypic analysis is performed and used to calculated the IC₅₀ or IC₉₀ of a drug for a virus variant. The results of the analysis can also be presented as fold-change in IC₅₀ or IC₉₀ for each variant as compared with a drug-susceptible reference virus or a viral sample taken from the same subject prior to a drug therapy.

Any method known in the art, without limitation, can be used to determine the phenotypic susceptibility or resistance of a mutant virus or population of viruses to an anti-viral therapy. Examples of determining phenotypes may found, for example, in U.S. Pat. Nos. 6,653,081, 6,489,098, 6,351,690, 6,242,187, 5,837,464, each of which is incorporated herein in its entirety for all purposes. For example, a phenotypic can be performed using the PHENOSENSE™ phenotype HIV assay (ViroLogic Inc., South San Francisco, Calif.). See Petropoulos et al., 2000, Antimicrob. Agents Chemother. 44:920-928, incorporated herein in its entirety for all purposes.

Any method known in the art can be used to determine whether a mutation is correlated with an increase in resistance of a virus to an protease inhibitor. Typically, P values are used to determine the statistical significance of the correlation, such that the smaller the P value, the more significant the measurement. Preferably the P values will be less than 0.05 (or 5%). More preferably, P values will be less than 0.01. P values can be calculated by any means known to one of skill in the art. For the purposes of correlating an increase in resistance of an HIV to a mutation, P values can be calculated using Fisher's Exact Test. See, e.g., David Freedman, Robert Pisani & Roger Purves, 1980, STATISTICS, W. W. Norton, New York. P values may be calculated using Student's paired and/or unpaired t-test and the non-parametric Kruskal-Wallis test (Statview 5.0 software, SAS, Cary, NC).

Resistance mutations in the HIV protease gene are generally classified into two groups. A first group typically includes those mutations either selected first in the presence of the drug or are otherwise shown to have an effect on drug binding to the protease or an effect on viral activity and replication. A second group of mutations may include mutations that appear later than primary mutations and by themselves do not have a significant effect on resistance phenotype. This second group of mutations are frequently thought to improve replicative fitness caused by mutations of the first group.

Section 6.1 below provides additional details on the identification of an optimum set of HIV protease mutations correlated to IDV/RTV resistance comprising a set of primary mutations (M46I/L/V, G48M/S/V, V82A/F/S/T, I84A/V, and L90M) and a set of secondary mutations (L10I/F/R/V, K20I/M/R/T, L24I, V32I, L33F, E34Q, M36I/L, K43T, I47A/V, I54A/L/M/S/T/V, L63P/S/A/T/Q/V/C, A71I/L/V/T, G73A/C/S/T, N88S/T, and L89V) that includes previously unrecognized mutations E34Q, K43T and L89V.

Thus in one aspect, the present invention provides a method for assessing the effectiveness of ritonavir-boosted indinavir therapy in a HIV-infected subject comprising determining whether a HIV from the subject contains a nucleic acid encoding HIV protease having one or more primary mutations where the one or more primary mutations are in the nucleic acid encoding codon 46, 48, 82, 84, or 90 of HIV protease, and one or more secondary mutations where the one or more secondary mutations are in the nucleic acid encoding codon 10, 20, 24, 32, 33, 34, 36, 43, 46, 47, 48, 54, 63, 71, 73, 82, 84, 88, 89, or 90 of HIV protease, in a combination of one primary mutation and at least six secondary mutations, or two primary mutations and at least four secondary mutations, or three or more primary mutations and at least one secondary mutation, wherein a mutation counted as a primary mutation may not also be counted as a secondary mutation, such that the presence of such a combination indicates a decrease in susceptibility to ritonavir-boosted indinavir, thereby assessing the effectiveness of ritonavir-boosted indinavir therapy in the subject.

Biological samples may include any sample that will contain an HIV. Biological samples from an HIV-infected subject include, for example and without limitation, blood, blood plasma, serum, urine, saliva, tissue swab and the like.

In an embodiment, the one or more primary mutations encode for an amino acid selected from the group consisting of M46I/L/V, G48M/S/V, V82A/F/S/T, I84A/V, and L90M, and the one or more secondary mutations encode for an amino acid selected from the group consisting of L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M.

Any method known to those of skill in the art may be used for detecting the presence or absence of a mutation in the protease of a HIV. The following section provides additional exemplary non-limiting guidance.

5.3.2 Detecting the Presence or Absence of Mutations in a Virus

The presence or absence of a viral mutation according to the present invention can be detected by any means known in the art for detecting a mutation. By “mutation” it is meant any variability in the nucleic acid sequence of a given HIV, or in the polypeptide sequence of the proteins of a given HIV, as compared to a reference HIV. Typically mutations of interest are those identified to confer resistance to a particular antiviral drug or combination of drugs, either existing alone or in a combination with other mutations. Thus, the mutation can be detected in the viral gene that encodes a particular protein, or in the protein itself, i.e., in the amino acid sequence of the protein.

In one embodiment, the mutation is in the viral nucleic acid. Such a mutation can be in, for example, a gene encoding a viral protein, in a cis or trans acting regulatory sequence of a gene encoding a viral protein, an intergenic sequence, or an intron sequence. The mutation can affect any aspect of the structure, function, replication or environment of the virus that changes its susceptibility to an anti-viral treatment. In one embodiment, the mutation is in a gene encoding a viral protein that is the target of an anti-viral treatment.

In another embodiment, the mutation is in a HIV nucleic acid encoding a protease. For example, the mutation can any mutation in codons 10, 20, 24, 32, 33, 34, 36, 43, 46, 47, 48, 54, 63, 71, 73, 82, 84, 88, 89, or 90. In one embodiment, the mutation in the nucleic acid encodes a mutant amino acid in a HIV protease selected from the group consisting of M46I/L/V, G48M/S/V, V82A/F/S/T, I84A/V, L90M, L10I/F/R/V, K20I/M/R/T, L24I, V32I, L33F, E34Q, M36I/L, K43T, I47A/V, I54A/L/M/S/T/V, L63P/S/A/T/Q/V/C, A71I/L/V/T, G73A/C/S/T, N88S/T, and L89V.

In an embodiment, the mutation in a HIV nucleic acid encodes a mutant amino acid in an HIV protease selected from the group consisting of M46I/L/V, G48M/S/V, V82A/F/S/T, I84A/V, and L90M.

In an embodiment, the mutation in the HIV nucleic acid encodes a mutant amino acid in the HIV protease selected from the group consisting of L10I/F/R/V, K20I/M/R/T, L24I, V32I, L33F, E34Q, M36I/L, K43T, I47A/V, I54A/L/M/S/T/V, L63P/S/A/T/Q/V/C, A71I/L/V/T, G73A/C/S/T, N88S/T, L89V.

In one embodiment, the mutation in a HIV nucleic acid confers a HIV phenotype resistant to indinavir.

In another embodiment, the mutation in a HIV nucleic acid confers a HIV phenotype resistant to ritonavir-boosted indinavir.

A mutation within a viral gene can be detected by utilizing a number of techniques. Viral DNA or RNA can be used as the starting point for such assay techniques, and may be isolated according to standard procedures which are well known to those of skill in the art.

The detection of a mutation in specific nucleic acid sequences, such as in a particular region of a viral gene, can be accomplished by a variety of methods including, but not limited to, restriction-fragment-length-polymorphism detection based on allele-specific restriction-endonuclease cleavage (Kan and Dozy, 1978, Lancet ii:910-912), mismatch-repair detection (Faham and Cox, 1995, Genome Res 5:474-482), binding of MutS protein (Wagner et aL., 1995, Nucl Acids Res 23:3944-3948), denaturing-gradient gel electrophoresis (Fisher et al., 1983, Proc. Natl. Acad. Sci. U.S.A. 80:1579-83), single-strand-conformation-polymorphism detection (Orita et al., 1983, Genomics 5:874-879), RNAase cleavage at mismatched base-pairs (Myers et aL., 1985, Science 230:1242), chemical (Cotton et al., 1988, Proc. Natl. Acad. Sci. U.S.A. 85:4397-4401) or enzymatic (Youil et al., 1995, Proc. Natl. Acad. Sci. U.S.A. 92:87-91) cleavage of heteroduplex DNA, methods based on oligonucleotide-specific primer extension (Syvänen et aL., 1990, Genomics 8:684-692), genetic bit analysis (Nikiforov et aL., 1994, Nucl Acids Res 22:4167-4175), oligonucleotide-ligation assay (Landegren et al., 1988, Science 241:1077), oligonucleotide-specific ligation chain reaction (“LCR”) (Barrany, 1991, Proc. Natl. Acad. Sci. U.S.A. 88:189-193), gap-LCR (Abravaya et al., 1995, Nucl Acids Res 23:675-682), radioactive or fluorescent DNA sequencing using standard procedures well known in the art, and peptide nucleic acid (PNA) assays (Orum et al., 1993, Nucl. Acids Res. 21:5332-5356; Thiede et al., 1996, Nucl. Acids Res. 24:983-984).

In addition, viral DNA or RNA may be used in hybridization or amplification assays to detect abnormalities involving gene structure, including point mutations, insertions, deletions and genomic rearrangements. Such assays may include, but are not limited to, Southern analyses (Southern, 1975, J. Mol. Biol. 98:503-517), single stranded conformational polymorphism analyses (SSCP) (Orita et al., 1989, Proc. Natl. Acad. Sci. USA 86:2766-2770), and PCR analyses (U.S. Pat. Nos. 4,683,202; 4,683,195; 4,800,159; and 4,965,188; PCR Strategies, 1995 Innis et al. (eds.), Academic Press, Inc.).

Such diagnostic methods for the detection of a gene-specific mutation can involve for example, contacting and incubating the viral nucleic acids with one or more labeled nucleic acid reagents including recombinant DNA molecules, cloned genes or degenerate samples thereof, under conditions favorable for the specific annealing of these reagents to their complementary sequences. Preferably, the lengths of these nucleic acid reagents are at least 15 to 30 nucleotides. After incubation, all non-annealed nucleic acids are removed from the nucleic acid molecule hybrid. The presence of nucleic acids which have hybridized, if any such molecules exist, is then detected. Using such a detection scheme, the nucleic acid from the virus can be immobilized, for example, to a solid support such as a membrane, or a plastic surface such as that on a microtiter plate or polystyrene beads. In this case, after incubation, non-annealed, labeled nucleic acid reagents of the type described above are easily removed. Detection of the remaining, annealed, labeled nucleic acid reagents is accomplished using standard techniques well-known to those in the art. The gene sequences to which the nucleic acid reagents have annealed can be compared to the annealing pattern expected from a normal gene sequence in order to determine whether a gene mutation is present.

Alternative diagnostic methods for the detection of gene specific nucleic acid molecules may involve their amplification, e.g., by PCR (U.S. Pat. Nos. 4,683,202; 4,683,195; 4,800,159; and 4,965,188; PCR Strategies, 1995 Innis et al. (eds.), Academic Press, Inc.), followed by the detection of the amplified molecules using techniques well known to those of skill in the art. The resulting amplified sequences can be compared to those which would be expected if the nucleic acid being amplified contained only normal copies of the respective gene in order to determine whether a gene mutation exists.

Additionally, the nucleic acid can be sequenced by any sequencing method known in the art. For example, the viral DNA can be sequenced by the dideoxy method of Sanger et al., 1977, Proc. Natl. Acad. Sci. USA 74:5463, as further described by Messing et al., 1981, Nuc. Acids Res. 9:309, or by the method of Maxam et al., 1980, Methods in Enzymology 65:499. See also the techniques described in Sambrook et al., 2001, Molecular Cloning: A Laboratory Manual, Cold Spring Harbor Laboratory, 3^{rd} ed., NY; and Ausubel et al., 1988 & updates, Current Protocols in Molecular Biology, John Wiley & Sons, NY.

The methods of the instant invention are applicable for determining resistance of an individual viral variant or for determining resistance of a variant population, which may be genotyped simultaneously. For example, for a given sequence, such as PR, sequencing a variant population together provides a genotype that can be used for identification of pertinent PR mutations.

Within the past decade, several technologies have been developed making it possible to identify large numbers (e.g., hundreds to hundreds of thousands) of nucleic acid sequences in a sample at any one time. See, e.g., Kozal et al., 1996, Nat. Med. 2(7):753-759; Lockhart et al., 1996, Nature Biotechnology 14:1675-1680; Blanchard et al., 1996, Nature Biotechnology 14, 1649; U.S. Pat. No. 5,571,639 issued Nov. 5, 1996. For example, a set oligonucleotide probes of predetermined sequences complimentary to various genotypes of a HIV protease can be attached to specific locations on a solid phase (an array), and the presence or absence of the various sequences in a unknown HIV nucleic acid sequence are determined by the hybridization patterns of the unknown HIV nucleic acid to the probes on the solid-phase. Typically, computer-aided techniques are used to assist in the gathering, processing, and evaluation of the large amount of information garnered in using array-based technology. See, e.g., U.S. Pat. No. 6,546,340 issued Apr. 8, 2003. Probe arrays including those made to custom specifications, along with reagents and computer analysis software are all commercially available (e.g., Affymetrix, Inc., Santa Clara Cailf.).

Identification of a mutation in an HIV protease may be determined by amino acid analysis of the protease. Identification of a mutation in an HIV protease may be determined by the use of antibodies specifically recognizing particular amino residues at certain positions in HIV protease. Such antibodies can be used in ELISA assays or immunoprecipitation studies to assess the presence of mutant amino acids in the protease.

5.3.3 Applying an Classification Rule to a Genotype

The methods of the present invention apply certain selection rules upon the identified HIV genotypes to classify a HIV as being resistant (or less susceptable) to a PRI or as being sensitive to a PRI.

In one embodiment, the selection rule requires a condition to be met that one primary mutation and at least six secondary mutations, or two primary mutations and at least four secondary mutations, or three or more primary mutation and at least one secondary mutation, where a primary mutation present in the HIV is counted as a secondary mutation only if it is not being counted as a primary mutation.

Any method known to those of skill in the art may employed to determined whether the conditions as applied to given HIV are met. Typically, computers are employed that perform the function of determining whether the genotype of an HIV meets the conditions for being classified as resistant to a drug. How computers are programmed to determine whether the conditions are met is not crucial to the practice of the instant invention as long as the conditions for selecting resistant genotypes are properly applied. Thus, any type of computer and any type programming language known to those of skill in the are can be employed that can determine if a HIV genotype meets a condition for being drug resistant.

In certain embodiment, methods for assessing the effectiveness of IDV/RTV therapy in a HIV-infected subject are provided that comprise determining whether a nucleic acid of a HIV of the subject contains a nucleic acid encoding HIV protease having
(i) one or more primary mutations where the one or more primary mutations are in the nucleic acid encoding codon 46, 48, 82, 84,or 90 of HIV protease, and
(ii) one or more secondary mutations where the one or more secondary mutations are in the nucleic acid encoding codon 10, 20, 24, 32, 33, 34, 36, 43, 46, 47, 48, 54, 63, 71, 73, 82, 84, 88, 89, or 90 of HIV protease,
in a combination of
   one primary mutation and at least six secondary mutations, or
   two primary mutations and at least four secondary mutations, or
   three or more primary mutations and at least one secondary mutation,
   wherein a mutation counted as a primary mutation may also not be counted as a secondary mutation.

Determining whether a nucleic acid contains one of the recited combination of mutations can be performed by any means known to those of skill in the art, without limitation. Any sequence of steps taken for making the determination, without limitation, may be taken so long as the recited combination can be determined. Thus, those of skill in the art recognize that no temporal order of steps for making the determination is intended by using the terms “primary mutation” and “secondary mutation” or by the order they are recited in an embodiment. For example, secondary mutations can be detected before primary mutations, or primary mutations can be detected before secondary mutations, or both primary and secondary mutations may be simultaneously detected.

As provided in Section 6, exemplary data indicates that the genotype interpretation algorithm can be applied in the methods of the invention for identifying an HIV that is resistant to IDV/RTV. Because the genotyping interpretation rules were developed using a relevant clinical cutoff value, those of skill in the art recognize the immediate benefit that the methods of the instant invention can have in addressing whether a given HIV will susceptible to IDV/RTV therapy in a subject infected with the HIV.

Thus, in certain embodiments, the identification of HIV as being resistant to IDV/RTV indicates a decrease in susceptibility to IDV/RTV therapy about equal to or greater than a clinical cutoff value of 10.

5.3.4 Correlating Phenotypic and Genotypic Susceptibility

Any method known in the art can be used to determine whether a mutation is correlated with a decrease in susceptibility of a virus to an anti-viral treatment and thus is a resistance-associated mutation (“RAM”) according to the present invention. In one embodiment, P values are used to determine the statistical significance of the correlation, such that the smaller the P value, the more significant the measurement. Preferably the P values will be less than 0.05. More preferably, P values will be less than 0.01. P values can be calculated by any means known to one of skill in the art. In one embodiment, P values are calculated using Fisher's Exact Test. See, e.g., David Freedman, Robert Pisani & Roger Purves, 1980, STATISTICS, W. W. Norton, New York.

In a preferred embodiment, numbers of samples with the mutation being analyzed that have an IC₅₀ fold change below or above 2.5-fold are compared to numbers of samples without the mutation. A 2×2 table can be constructed and the P value can be calculated using Fisher's Exact Test. In such embodiments, P values smaller than 0.05 or 0.01 can be classified as statistically significant.

5.4 Constructing an Algorithm

In another aspect, the present invention provides a method of constructing an algorithm that correlates genotypic data about a virus with phenotypic data about the virus. In certain embodiments, the phenotypic data relate to the susceptibility of the virus to an anti-viral treatment. In certain embodiments, the anti-viral treatment is an anti-viral compound. In certain embodiments, the anti-viral compound is a protease inhibitor. In certain embodiments, the protease inhibitor is ritonavir. In certain embodiments, the protease inhibitor is a combination of ritonavir and indinavir.

In one embodiment, the method of constructing the algorithm comprises creating a rule or rules that correlate genotypic data about a set of viruses with phenotypic data about the set of viruses.

In one embodiment, a data set comprising genotypic and phenotypic data about each virus in a set of viruses is assembled. Any method known in the art can be used to collect genotypic data about a virus. Examples of methods of collecting such data are provided below. Any method known in the art can be used for collecting phenotypic data about a virus. Examples of such methods are provided below. In a preferred embodiment, the data set comprises one or more RAMs as described above. In certain embodiments, each genotypic datum is the sequence of all or part of a viral protein of a virus in the set of viruses. In certain embodiments, each genotypic datum in the data set is a single amino acid change in a protein encoded by the virus, relative to a reference protein in the reference virus. In certain embodiments, the genotype comprises two, three, four, five, six or more amino acid changes in the viral protein. In certain embodiments, the virus is HW, and the protein is HIV protease. In a preferred embodiment, the virus is HIV-1. In certain embodiments, the reference protein is the protease from NL4-3 HIV.

In certain embodiments, each phenotypic datum in the data set is the susceptibility to an anti-viral treatment of a virus in the set of viruses. In certain embodiments, the anti-viral treatment is an anti-viral compound. In certain embodiments, the anti-viral compound is a protease inhibitor. In a preferred embodiment, the protease inhibitor is RTV-boosted indinavir. In certain embodiments, the susceptibility is measured as a change in the susceptibility of the virus relative to a reference virus. In certain embodiments, the susceptibility is measured as a change in the IC₅₀ of the virus relative to a reference virus. In certain embodiments, the change in IC₅₀ is represented as the fold-change in IC₅₀. In certain embodiments, the virus is HIV. In a preferred embodiment, the virus is HIV-1. In another preferred embodiment, the reference HIV is NL4-3 HIV.

The genotypic and phenotypic data in the data set can be represented or organized in any way known in the art. In certain embodiments, the data are displayed in the form of a graph. In certain embodiments of this type of representation, the y-axis represents the fold change in IC₅₀ of a virus in the data set relative to a reference virus. In certain embodiments, each point on the graph corresponds to one virus in the data set. In certain embodiments, the x-axis represents the number of mutations that a virus in the data set has. In certain embodiments, the position of the point indicates both the number of mutations and the fold change in anti-viral therapy treatment that the virus has, both measured relative to a reference strain. In certain embodiments, the genotypic and phenotypic data in the data set are displayed in the form of a chart.

In one aspect, an algorithm is formulated that correlates the genotypic data with the phenotypic data in the data set. In certain embodiments, a phenotypic cutoff point is defined. In a preferred embodiment, the phenotype is susceptibility to an anti-viral treatment. In certain embodiments, the phenotype is change in sensitivity to an anti-viral treatment relative to a reference virus, and the cutoff point is the value above which a virus or population of viruses is defined as phenotypically resistant (“PT-R”) to the anti-viral therapy and below which a virus or population of viruses is defined as phenotypically sensitive (“PT-S”) to the anti-viral therapy. In certain embodiments, the cutoff point is 2-fold, 2.5-fold, 3-fold, 5-fold, 10-fold, 15-fold, 20-fold, 30-fold, 40-fold, 50-fold or 100-fold greater than the IC₅₀ of a reference virus. In certain embodiments, the phenotypic cutoff point is the clinical cutoff value as defined above. In a preferred embodiment, the virus is HIV and the anti-viral therapy is treatment with a protease inhibitor. In a preferred embodiment, the protease inhibitor is RTV-boosted indinavir.

In certain embodiments, the phenotypic cutoff point is used to define a genotypic cutoff point. In certain embodiments, this is done by correlating the number of mutations in a virus of the data set with the phenotypic susceptibility of the virus. This can be done, for example, using a graph similar to one discussed above. A genotypic cutoff point can be selected such that most viruses having more than that number of mutations in the data set are phenotypically resistant (“PT-R”), and most viruses having fewer than that number of mutations are phenotypically sensitive (“PT-S”). By definition, a virus in the data set with number of mutations equal to, or more than the genotypic cutoff is genotypically resistant (“GT-R”) to the anti-viral treatment, and a virus in the data set with fewer than the genotypic cutoff number of mutations is genotypically sensitive (“GT-S”) to the anti-viral treatment. Thus, in certain embodiments, a genotypic cutoff point is selected that produces the greatest percentage of viruses in the data set that are either phenotypically resistant and genotypically resistant (“PT-R, GT-R”), or phenotypically sensitive and genotypically sensitive (“PT-S, GT-S”).

While this algorithm can provide a useful approximation of the relationship between the genotypic and phenotypic data in the data set, in most cases there will be a significant number of strains that are genotypically sensitive but phenotypically resistant (“GT-S, PT-R”), or genotypically resistant but phenotypically sensitive (“GT-R, PT-S”). Thus, in a preferred embodiment, the algorithm is further modified to reduce the percentage of discordant results in the data set. This can be done, for example, by removing from the data set each data point that corresponds to a virus population comprising a mixture of mutations including the wild-type, at a single position considered by the algorithm tested. This can have the effect of reducing the number of PT-S, GT-R results, thus lowering the overall percentage of discordant results and so improves the fit of the algorithm to a data set.

In certain embodiments, differential weight values are assigned to one or more mutations observed in the data set. An algorithm that does not include this step assumes that each mutation in the data set contributes equally to the overall resistance of a virus or population of viruses to an anti-viral therapy. For example, a mutation could be present in a data set that is almost always correlated with phenotypic resistance to an anti-viral treatment. That is, almost every virus that has the mutation is phenotypically resistant to the anti-viral treatment, even those strains having only one or two total mutations. In certain embodiments, such mutations are “weighted,” i.e., assigned an increased mutation score. A mutation can be assigned a weight of, for example, two, three, four, five, six, seven, eight or more. For example, a mutation assigned a weight of 2 will be counted as two mutations in a virus. Fractional weighting values can also be assigned. In certain embodiments, values of less than 1, and of less than zero, can be assigned, wherein a mutation is associated with an increased sensitivity of the virus to the anti-viral treatment.

One of skill in the art will appreciate that there is a tradeoff involved in assigning an increased weight to certain mutations. As the weight of the mutation is increased, the number of GT-R, PT-S discordant results may increase. Thus, assigning a weight to a mutation that is too great may increase the overall discordance of the algorithm. Accordingly, in certain embodiments, a weight is assigned to a mutation that balances the reduction in GT-S, PT-R discordant results with the increase in GT-R, PT-S discordant results.

In certain embodiments, the interaction of different mutations in the data set with each other is also factored into the algorithm. For example, it might be found that two or more mutations behave synergistically, i.e., that the coincidence of the mutations in a virus contributes more significantly to the resistance of the virus than would be predicted based on the effect of each mutation independent of the other. Alternatively, it might be found that the coincidence of two or more mutations in a virus contributes less significantly to the resistance of the virus than would be expected from the contributions made to resistance by each mutation when it occurs independently. Also, two or more mutations may be found to occur more frequently together than as independent mutations. Thus, in certain embodiments, mutations occurring together are weighted together. For example, only one of the mutations is assigned a weight of 1 or greater, and the other mutation or mutations are assigned a weight of zero, in order to avoid an increase in the number of GT-R, PT-S discordant results.

In another aspect, the phenotypic cutoff point can be used to define a genotypic cutoff point by correlating the number as well as the class of mutations in a virus of the data set with the phenotypic susceptibility of the virus. Examples of classes of mutations include, but are not limited to, primary amino acid mutations, secondary amino acid mutations, mutations in which the net charge on the polypeptide is conserved and mutations that do not alter the polarity, hydrophobicity or hydrophilicity of the amino acid at a particular position. Other classes of mutations that are within the scope of the invention would be evident to one of skill in the art, based on the teachings herein.

In certain embodiments, an algorithm is constructed that factors in the requirement for one or more classes of mutations. In certain embodiments, the algorithm factors in the requirement for a minimum number of one or more classes of mutations. In certain embodiments, the algorithm factors in the requirement for a minimum number of primary or secondary mutations. In certain embodiments, the requirement for a primary or a secondary mutation in combination with other mutations is also factored into the algorithm. For example, it might be found that a virus with a particular combination of mutations is resistant to an anti-viral treatment, whereas a virus with any mutation in that combination, alone or with other mutations that are not part of the combination, is not resistant to the anti-viral treatment.

By using, for example, the methods discussed above, the algorithm can be designed to achieve any desired result. In certain embodiments, the algorithm is designed to maximize the overall concordance (the sum of the percentages of the PT-R, GT-R and the PT-S, GT-S groups, or 100 minus (percentage of the PT-S, GT-R+PT-R, GT-S groups). In preferred embodiments, the overall concordance is greater than about 75%, 80%, 85%, 90% or 95%. In certain embodiments, the algorithm is designed to minimize the percentage of PT-R, GT-S results. In certain embodiments, the algorithm is designed to minimize the percentage of PT-S, GT-R results. In certain embodiments, the algorithm is designed to maximize the percentage of PT-S, GT-S results. In certain embodiments, the algorithm is designed to maximize the percentage of PT-R, GT-R results.

At any point during the construction of the algorithm, or after it is constructed, it can be further tested on a second data set. In certain embodiments, the second data set consists of viruses that are not included in the data set used to construct the algorithm, i.e., the second data set is a naive data set. In certain embodiments, the second data set contains one or more viruses that were in the data set used to construct the algorithm and one or more viruses that were not in that data set. Use of the algorithm on a second data set, particularly a naive data set, allows the predictive capability of the algorithm to be assessed. Thus, in certain embodiments, the accuracy of an algorithm is assessed using a second data set, and the rules of the algorithm are modified as described above to improve its accuracy. In a preferred embodiment, an iterative approach is used to create the algorithm, whereby an algorithm is tested and then modified repeatedly until a desired level of accuracy is achieved.

In one aspect, the construction or implementation of the algorithm can begin with a few “starting mutations” and proceed in steps in which it factors in the presence of certain mutations or classes of mutations. In one embodiment, the algorithm factors in the presence of one or more primary mutations, as described above, plus two secondary mutations. Any of the mutations listed in Table 1 can be used as secondary mutations. Next, the algorithm factors in other mutations in addition to the starting mutations. In certain embodiments, the algorithm, in all future stages, factors in a minimum number of secondary mutations. In a more particular embodiment, the algorithm, in all future stages, factors in at least 2 secondary mutations. When the algorithm factors in the combination of 2 or more mutations, it is generally understood that both mutations, e.g., 33F and 82A, be present in the same virus (or sample). Finally, the algorithm can factor in additional combinations, e.g., the combination of 46I or 46L with any one or more of 47V, 54V, 71L, 76V, or 82A. During the construction or implementation of an algorithm as described above, a decrease in the overall discordance as well as the percentage of data in the PT-R, GT-S group decreased with each step of the algorithm is indicative that the algorithm improved each time in correctly predicting the mutations and combinations of mutations that led to phenotypic resistance.

5.5 Using an Algorithm to Predict the Susceptibility of a Virus

In another aspect, the present invention also provides a method for using an algorithm of the invention to predict the phenotypic susceptibility of a virus or a derivative of a virus to an anti-viral treatment based on the genotype of the virus. In one embodiment, the method comprises detecting, in the virus or derivative of the virus, the presence or absence of one or more RAMs, applying the rules of the algorithm to the detected RAMs, wherein a virus that satisfies the rules of the algorithm is genotypically resistant to the anti-viral treatment, and a virus that does not satisfy the rules of the algorithm is genotypically sensitive to the anti-viral treatment. In another embodiment, the method comprises detecting, in the virus or derivative of the virus, the presence or absence of one or more RAMs, applying the rules of the algorithm to the detected RAMs, wherein a score equal to, or greater than the genotypic cutoff score indicates that the virus is genotypically resistant to the anti-viral treatment, and a score less than the genotypic cutoff score indicates that the virus is genotypically sensitive to the anti-viral treatment.

The algorithm of this invention can be used for any viral disease where anti-viral drug susceptibility is a concern, as discussed herein. In certain embodiments the assay of the invention can be used to determine the susceptibility of a retrovirus to an anti-viral drug. In a preferred embodiment, the retrovirus is HIV. Preferably, the virus is HIV-1.

The anti-viral agent of the invention could be any treatment effective against a virus. It is useful to the practice of this invention, for example, to understand the structure, life cycle and genetic elements of the viruses which can be tested in the drug susceptibility test of this invention. These would be known to one of ordinary skill in the art and provide, for example, key enzymes and other molecules at which the anti-viral agent can be targeted. Examples of anti-viral agents of the invention include, but are not limited to, nucleoside reverse transcriptase inhibitors such as AZT, ddI, ddC, d4T, 3TC, abacavir, nucleotide reverse transcriptase inhibitors such as tenofovir, non-nucleoside reverse transcriptase inhibitors such as nevirapine, efavirenz, delavirdine, fusion inhibitors such as T-20 and T-1249 and protease inhibitors such as saquinavir, ritonavir, indinavir, nelfinavir, amprenavir and lopinavir.

In some embodiments of the invention, the anti-viral agents are directed at retroviruses. In certain embodiments, the anti-viral agents are protease inhibitors such as saquinavir, ritonavir, indinavir, nelfinavir, amprenavir and lopinavir. In certain embodiments, the anti-viral agents comprise two or more protease inhibitors. In certain embodiments, the protease inhibitors are administered in combination. In a preferred embodiment, the anti-viral agents are ritonavir and indinavir.

Some mutations associated with reduced susceptibility to treatment with an anti-viral agent are known in the art. See, e.g., Maguire et al., 2002, Antimicrob Agents Chemother 46:731-738. Others can be determined by methods described herein. For example, Table 1 provides a list of mutations associated with reduced susceptibility to RTV-boosted indinavir.

5.6 Using an Algorithm to Predict the Effectiveness of Anti-Viral Treatment for an Individual

In another aspect, the present invention also provides a method for using an algorithm of the invention to predict the effectiveness of an anti-viral treatment for an individual infected with a virus based on the genotype of the virus to the anti-viral treatment. In certain embodiments, the method comprises detecting, in the virus or derivative of the virus, the presence or absence of one or more RAMs, applying the rules of the algorithm to the detected RAMs, wherein a virus that satisfies the rules of the algorithm is genotypically resistant to the anti-viral treatment, and a virus that does not satisfy the rules of the algorithm is genotypically sensitive to the anti-viral treatment, thereby identifying the effectiveness of the anti-viral treatment. In certain embodiments, the method comprises detecting, in the virus or a derivative of the virus, the presence or absence of one or more RAMs, applying the rules of the algorithm to the detected RAMs, wherein a score equal to, or greater than the genotypic cutoff score indicates that the virus is genotypically resistant to the anti-viral treatment, and a score less than the genotypic cutoff score indicates that the virus is genotypically sensitive to the anti-viral treatment.

As described in above, the algorithm of the invention can be used for any viral disease where anti-viral drug susceptibility is a concern and the anti-viral agent of the invention could be any treatment effective against a virus. In certain embodiments the assay of the invention is used to determine the susceptibility of a retrovirus to an anti-viral drug. In a preferred embodiment, the retrovirus is HIV. Preferably, the virus is HIV-1. In some embodiments of the invention, the anti-viral agents are directed at retroviruses. In certain embodiments, the anti-viral agents are protease inhibitors such as saquinavir, ritonavir, indinavir, nelfinavir, amprenavir and lopinavir. In certain embodiments, the anti-viral agents comprise two or more protease inhibitors. In certain embodiments, the protease inhibitors are administered in combination. In a preferred embodiment, the anti-viral agents are ritonavir and indinavir.

As described above, mutations associated with reduced susceptibility to treatment with an anti-viral agent may be obtained from the art or determined by methods described herein.

In certain embodiments, the present invention provides a method for monitoring the effectiveness of an anti-viral treatment in an individual infected with a virus and undergoing or having undergone prior treatment with the same or different anti-viral treatment. In certain embodiments, the method comprises detecting, in a sample of the individual, the presence or absence of an amino acid residue associated with reduced susceptibility to treatment the anti-viral treatment, wherein the presence of the residue correlates with a reduced susceptibility to treatment with the anti-viral treatment.

5.7 Correlating Susceptibility to one Anti-Viral Treatment with Susceptibility to Another Anti-Viral Treatment

In another aspect, the present invention provides a method for using an algorithm of the invention to predict the effectiveness of an anti-viral treatment against a virus based on the genotypic susceptibility of the virus to a different anti-viral treatment. In certain embodiments, the method comprises detecting, in a virus or a derivative of a virus, the presence or absence of one or more mutations correlated with resistance to an anti-viral treatment and applying the rules of an algorithm of the invention to the detected mutations, wherein a virus that satisfies the rules of the algorithm is genotypically resistant to the anti-viral treatment, and a virus that does not satisfy the rules of the algorithm is genotypically sensitive to the anti-viral treatment. In certain embodiments, the method comprises detecting, in the virus or a derivative of the virus, the presence or absence of one or more mutations correlated with resistance to an anti-viral treatment and applying the rules of the algorithm to the detected mutations, wherein a score equal to, or greater than the genotypic cutoff score indicates that the virus is genotypically resistant to a different anti-viral treatment, and a score less than the genotypic cutoff score indicates that the virus is genotypically sensitive to a different anti-viral treatment. In certain embodiments, the two anti-viral treatments affect the same viral protein. In certain embodiments, the two anti-viral treatments are both protease inhibitors. Examples of protease inhibitors include, but are not limited to, saquinavir, ritonavir, indinavir, nelfinavir, amprenavir and lopinavir. In certain embodiments, one of the two anti-viral treatments is indinavir. In certain embodiments, one of the two anti-viral treatments is ritonavir. In certain embodiments, a mutation correlated with resistance to one protease inhibitor is also correlated with resistance to another protease inhibitor.

5.8 Computer Implemented Methods

In one aspect, the present invention provides a computer implemented method of identifying a HIV as being less susceptible to ritonavir-boosted indinavir therapy in a subject infected with the HIV. Typically, data representing the HIV genotype is received as input by a computer system. For example, data can be entered by a keyboard. As another example, data can be received electronically from a device used for the purpose of genotyping nucleic acid. Typically genotyping of HIV nucleic acid is resolved by electrophoretic methods using dye termination chemistry reactions, although other options are possible including hybridization patterns of a HIV nucleic acid to oligonucleotide array. Thus, the data received as input may represent electrophoretic migrations or hybridization patterns which can be converted into a representation of a genotype.

Embodiments of the computer implemented method comprise performing comparison of the genotype of the HIV to a database representing pertinent protease inhibitor resistance mutations. In preferred embodiments, the database comprises representations of mutant codons L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M. Performing a comparison between the genotype of the HIV and the database can be performed in any sequential order, without limitation, and does not depend on considerations such amino acid position in the protease or whether a particular position represents a site of a primary or secondary mutation; it is only required that performing a comparison is undertaken in such a way that the recited conditions can be determined.

In one embodiment, a computer implemented method comprises determining whether a condition is met that one match is made in the first comparison and at least six matches are made in the second comparison; or two matches are made in the first comparison and at least four matches are made in the second comparison; or three or more matches are made in the first comparison and at least one match is made in the second comparison; with the proviso that a match made in the first comparison may not also be counted as a match in the second comparison.

The computer implemented methods disclosed herein may implemented on any computer that is known to those of skill in the art, without limitation. It will be recognized that the implemented methods disclosed herein do not depend on a particular type of computer, memory storage elements, processing speeds, programming languages, compilers, other computer hardware, software or peripherals, and the like.

In certain embodiments, the computer implemented methods comprise displaying a result indicating whether or not that the HIV is identified as being less susceptible to ritonavir-boosted indinavir therapy in a subject infected with the HIV. It is generally understood that an output device is used for the display of the results obtained using the computer-implemented methods of the invention. Output devices can be any type of printers, computer screens, disk drives, CD burners, other computers, or memory modules accessible by another computer, and the like without limitation. Displaying a result can be any display known to those of skill in the art without limitation.

In one embodiment, the result is displayed on a tangible medium. Typically, results are displayed on computer screens, printouts, CDs, and the like.

5.9 Other Methods

Those of skill in the art recognize the value of providing the information that can be obtained using the methods disclosed herein. For example, costly yet ineffective antiviral drug treatment regimens can be avoided with the knowledge that an HIV is resistant to a PRI.

In one aspect, the present invention provides a system of providing information of whether a HIV taken from a HIV-infected subject is resistant to ritonavir-boosted indinavir. This information may provided to the subject or to a health care professional.

Typically, the system comprises identifying primary and secondary mutations in a HIV and determining if the HIV is resistant to PRI using the algorithms disclosed herein.

In one embodiment, the method comprises obtaining a genotype for nucleic acid encoding HIV protease of the HIV This can be performed, for example, by receiving a HIV taken from the HIV-infected subject and determining the genotype of the protease using techniques as described herein or can be received from another who performed the genotyping on the HIV.

In another embodiment, the method comprises identifying the presence or absence of a primary mutation in the HIV comprising M46I/L/V, G48M/S/V, V82A/F/S/T, I84A/V, or L90M of HIV protease and identifying the presence or absence of a secondary mutation in the HIV comprising L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, or L90M of HIV protease. As previously explained, identifying the presence or absence of a primary or secondary mutation can be performed simultaneously or in any order.

In one embodiment, the method comprises determining whether a condition is met that the presence of one primary mutation and at least six secondary mutations are identified, or the presence of two primary mutations and at least four secondary mutations are identified, or three or more primary mutations and at least one secondary mutation are identified such that if a condition is met, then the HIV taken from the HIV-infected subject is resistant to ritonavir-boosted indinavir.

In one embodiment, the method comprises preparing a tangible medium comprising an indication of whether or not the HIV is resistant to ritonavir-boosted indinavir.

In one embodiment, the method comprises conveying the tangible medium to the subject or the health care provider.

5.10 Devices and Systems

In another aspect, the present invention provides a computer system that is configured to perform the computer implemented methods described in Section 5.8. Computers are particular helpful in the performance of the instant methods given the amount of genotype data in combination with rapidity of computers in performing algorithms.

In one embodiment, the computer system comprises a desktop computer running Microsoft WINDOWS operating system.

In another embodiment, the computer system comprises software written in PERL.

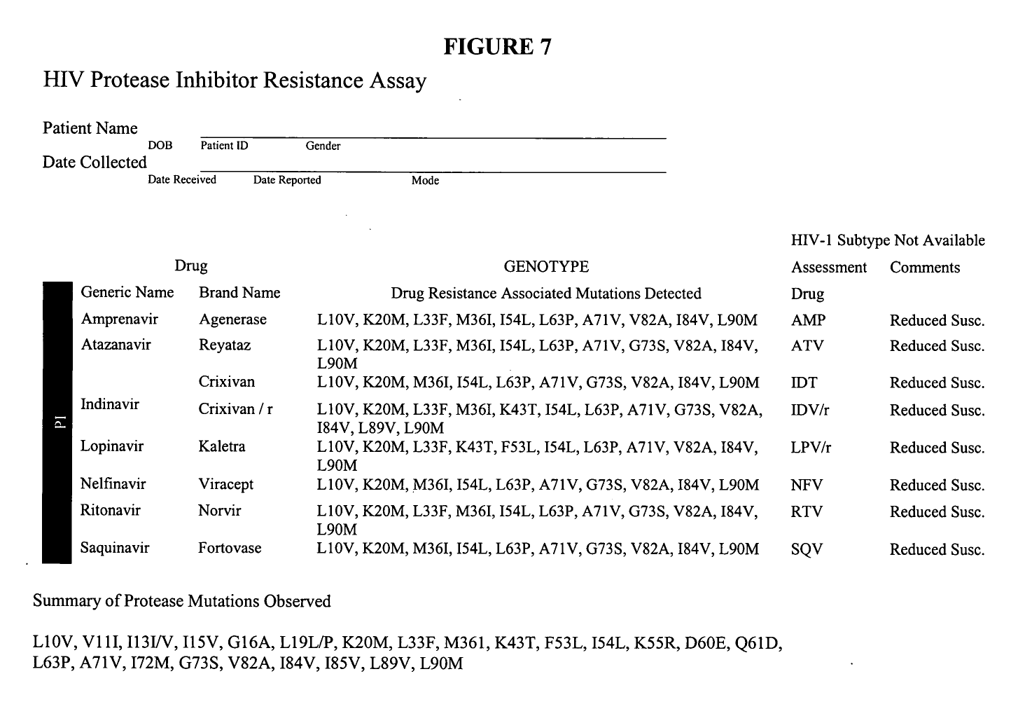

In another aspect, the present invention provides a paper display of the result produced by the methods disclosed herein. FIG. 7 depicts an representative paper display of a result produced by an exemplary method of the invention.

In yet another aspect, the invention provides an article of manufacture that comprises computer-readable instructions for performing the computer-implemented methods discussed above. One embodiment is a CD. Another embodiment is an CD wherein the computer-readable instructions are in PERL.

In another aspect, the present invention provides a computer program product comprising one or more computer codes that identify a HIV as being less susceptible to ritonivir-boosted indinavir drug treatment in a subject infected with HIV and a computer readable medium that stores the computer codes. Several embodiments follow.

In one embodiment, the computer program comprises a computer code that receives input corresponding to the genotype of the HIV nucleic acid encoding HIV protease. The input may represent the nucleotide sequence of the HIV nucleic acid, for example, a list of bases. The input may be converted from a hybridization pattern of the HIV nucleic acid onto an oligonucleotide probe array attached to a solid phase. The input may be converted from an automated sequencer detecting electrophoretic migration.

In another embodiment, the computer program comprises a computer code that performs a first comparison to determine if an amino acid encoded by HIV protease codons 46, 48, 82, 84 and 90 of the HIV nucleic acid matches one or more of mutant amino acids M46I/L/V, G48M/S/V, V82A/F/S/T, I84A/V, and L90M of HIV protease, and a computer code that performs a second comparison to determine if an amino acid encoded by HIV protease codons 10, 20, 24, 32, 33, 34, 36, 43, 46, 47, 48, 54, 63, 71, 73, 82, 84, 88, 89 and 90 of the HIV nucleic acid matches one or more of mutant amino acids L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M.

In another embodiment, the computer program comprises a computer code that determines whether a condition is met that one match is made in the first comparison and at least six matches are made in the second comparison, or two matches are made in the first comparison and at least four matches are made in the second comparison, or three or more matches are made in the first comparison and at least one match is made in the second comparison, with the proviso that a match made in the first comparison may not be counted as a match in the second comparison, wherein the HIV is identified as being less susceptible to ritonivir-boosted indinavir drug treatment in a subject infected with HIV if a condition is determined to be met.

In another embodiment, the computer program comprises a computer code conveys a result representing whether or not the HIV is identified as being less susceptible to ritonivir-boosted indinavir drug treatment in a subject infected with HIV to an output device. An output device may any known to those of skill in the art, without limitation, such as a printer, a disk drive, a computer screen, another computer, and so forth.

In an aspect, the present invention provides a tangible medium storing the result conveyed to an output device as described above. A tangible medium may be any tangible medium known to those of skill in the art without limitation. A tangible medium may be a CD or DVD. A tangible medium may be a printout.

### 6. EXAMPLES

### 6.1 Example 1

### Defining an Optimum Set of Protease Mutations and Numbers of Mutations to be Considered

A optimized set of protease mutations for IDV/RTV was generated utilizing the HIPAA-compliant database of over 26,000 linked phenotype and genotype results for patient's samples maintained by ViroLogic, Inc. (South San Francisco, Cailf.). Phenotypes and genotypes were determined in the Clinical Laboratory Improvement Amendments-approved ViroLogic clinical reference laboratory. The drug susceptibility phenotypes of HIV-1 isolates from patient plasma samples was determined by the PHENOSENSE™ phenotype HIV assay. This assay is performed by amplifying the PR-RT segment of the pol gene from patient plasma and inserting it into a genomic HIV-1 vector. The vector contains a luciferase reporter gene to monitor recombinant virus infection in cell culture. Results are expressed as the FC in the IC₅₀ for the patient-derived virus compared to that for a reference control virus, NL4-3. Drug dilutions are arranged to maximize curve-fitting accuracy for the range of wildtype virus susceptibilities over clinically relevant ranges of increased and decreased susceptibilities. Microtiter plates are incubated in customized incubators in which the termperature, CO₂ level, and humidity are controlled to minimize variation in cell growth and medium composition changes throughout the plate.

Genotypes were determined by the GENESEQ™ HIV assay. This assay uses the resistance test vectors constructed for the phenotype assay as the template, dye-terminator reaction chemistry, and automated capillary electrophoresis to determine the sequences of the patient-derived HIV-1 PRs (amino acids 1 to 99). The deduced amino acids sequences of patient viruses were compared to the sequence of NL4-3 (GenBank accession no. AF324493).

To determine an optimized set of protease mutations for IDV/RTV genotypic patterns associated with reductions in susceptibility of 10-fold or greater (clinical cutoff value) were determined. Univariate (Fisher exact test), classification tree (CART), and receiver-operator analyses were performed identify the optimum set of protease mutations and numbers of mutations to be considered. Table 1 presents a partial summary of data obtained in an analysis of genotypes to pertinent resistance mutations.

TABLE 1
Protease Mutations Associated with Reduced Susceptibility to IDV/RTV

| | FC <10 | | FC >10 | | N | P | | | |
|---|---|---|---|---|---|---|---|---|---|
| Mutation | mt | wt | mt | wt | mutant | value | mtS(%) | mtR(%) | OR |
| G48M* | 1 | 4525 | 67 | 3407 | 68 | <0.001 | 0.02% | 1.93% | 87.3 |
| I54S** | 2 | 4524 | 77 | 3397 | 79 | <0.001 | 0.04% | 2.22% | 50.2 |
| I54A** | 3 | 4523 | 108 | 3366 | 111 | <0.001 | 0.07% | 3.11% | 46.9 |
| I54T** | 8 | 4518 | 91 | 3383 | 99 | <0.001 | 0.18% | 2.62% | 14.8 |
| I13M | 4 | 4522 | 41 | 3433 | 45 | <0.001 | 0.09% | 1.18% | 13.4 |
| C67F | 14 | 4512 | 137 | 3337 | 151 | <0.001 | 0.31% | 3.94% | 12.7 |
| T91A | 6 | 4520 | 52 | 3422 | 58 | <0.001 | 0.13% | 1.50% | 11.3 |
| I84A* | 1 | 4525 | 8 | 3466 | 9 | 0.013 | 0.02% | 0.23% | 10.4 |
| A71L** | 7 | 4519 | 52 | 3422 | 59 | <0.001 | 0.15% | 1.50% | 9.7 |
| V11L | 12 | 4514 | 82 | 3392 | 94 | <0.001 | 0.27% | 2.36% | 8.9 |
| V82S* | 16 | 4510 | 104 | 3370 | 120 | <0.001 | 0.35% | 2.99% | 8.5 |
| L89V^{†} | 64 | 4462 | 345 | 3129 | 409 | <0.001 | 1.41% | 9.93% | 7.0 |
| C95F | 34 | 4492 | 179 | 3295 | 213 | <0.001 | 0.75% | 5.15% | 6.9 |
| T91S | 26 | 4500 | 132 | 3342 | 158 | <0.001 | 0.57% | 3.80% | 6.6 |
| V11I | 66 | 4460 | 331 | 3143 | 397 | <0.001 | 1.46% | 9.53% | 6.5 |
| E34K | 8 | 4518 | 39 | 3435 | 47 | <0.001 | 0.18% | 1.12% | 6.4 |
| G73T** | 70 | 4456 | 337 | 3137 | 407 | <0.001 | 1.55% | 9.70% | 6.3 |
| G48V* | 86 | 4440 | 366 | 3108 | 452 | <0.001 | 1.90% | 10.54% | 5.5 |
| K43T^{†} | 140 | 4386 | 566 | 2908 | 706 | <0.001 | 3.09% | 16.29% | 5.3 |
| G16A | 51 | 4475 | 206 | 3268 | 257 | <0.001 | 1.13% | 5.93% | 5.3 |
| I47A | 4 | 4522 | 16 | 3458 | 20 | 0.001 | 0.09% | 0.46% | 5.2 |
| V82F* | 40 | 4486 | 153 | 3321 | 193 | <0.001 | 0.88% | 4.40% | 5.0 |
| E34Q^{†} | 66 | 4460 | 252 | 3222 | 318 | <0.001 | 1.46% | 7.25% | 5.0 |
| C95L | 3 | 4523 | 11 | 3463 | 14 | 0.012 | 0.07% | 0.32% | 4.8 |
| G48Q* | 3 | 4523 | 10 | 3464 | 13 | 0.022 | 0.07% | 0.29% | 4.3 |
| I47V | 144 | 4382 | 454 | 3020 | 598 | <0.001 | 3.18% | 13.07% | 4.1 |
| I66V | 45 | 4481 | 138 | 3336 | 183 | <0.001 | 0.99% | 3.97% | 4.0 |
| P79D | 10 | 4516 | 30 | 3444 | 40 | <0.001 | 0.22% | 0.86% | 3.9 |
| I54V** | 697 | 3829 | 1993 | 1481 | 2690 | <0.001 | 15.40% | 57.37% | 3.7 |
| K55R | 201 | 4325 | 568 | 2906 | 769 | <0.001 | 4.44% | 16.35% | 3.7 |
| L241** | 161 | 4365 | 442 | 3032 | 603 | <0.001 | 3.56% | 12.72% | 3.6 |
| L76V | 71 | 4455 | 190 | 3284 | 261 | <0.001 | 1.57% | 5.47% | 3.5 |
| V321** | 178 | 4348 | 475 | 2999 | 653 | <0.001 | 3.93% | 13.67% | 3.5 |
| G73C** | 46 | 4480 | 121 | 3353 | 167 | <0.001 | 1.02% | 3.48% | 3.4 |
| V82M | 5 | 4521 | 13 | 3461 | 18 | 0.017 | 0.11% | 0.37% | 3.4 |
| F53L | 170 | 4356 | 435 | 3039 | 605 | <0.001 | 3.76% | 12.52% | 3.3 |
| P79A | 30 | 4496 | 76 | 3398 | 106 | <0.001 | 0.66% | 2.19% | 3.3 |
| I66F | 52 | 4474 | 128 | 3346 | 180 | <0.001 | 1.15% | 3.68% | 3.2 |
| V82T* | 116 | 4410 | 283 | 3191 | 399 | <0.001 | 2.56% | 8.15% | 3.2 |
| I54M** | 129 | 4397 | 313 | 3161 | 442 | <0.001 | 2.85% | 9.01% | 3.2 |
| V820 | 24 | 4502 | 54 | 3420 | 78 | <0.001 | 0.53% | 1.55% | 2.9 |
| D60N | 8 | 4518 | 18 | 3456 | 26 | 0.009 | 0.18% | 0.52% | 2.9 |
| I84V* | 647 | 3879 | 1414 | 2060 | 2061 | <0.001 | 14.30% | 40.70% | 2.8 |
| G73S** | 370 | 4156 | 787 | 2687 | 1157 | <0.001 | 8.17% | 22.65% | 2.8 |
| K70E | 30 | 4496 | 60 | 3414 | 90 | <0.001 | 0.66% | 1.73% | 2.6 |
| K20R** | 438 | 4088 | 856 | 2618 | 1294 | <0.001 | 9.68% | 24.64% | 2.5 |
| L33F | 559 | 3967 | 1055 | 2419 | 1614 | <0.001 | 12.35% | 30.37% | 2.5 |
| V82A* | 906 | 3620 | 1698 | 1776 | 2604 | <0.001 | 20.02% | 48.88% | 2.4 |
| I85V | 222 | 4304 | 408 | 3066 | 630 | <0.001 | 4.90% | 11.74% | 2.4 |
| K20I** | 316 | 4210 | 559 | 2915 | 875 | <0.001 | 6.98% | 16.09% | 2.3 |
| Q58E | 290 | 4236 | 511 | 2963 | 801 | <0.001 | 6.41% | 14.71% | 2.3 |
| Q18H | 91 | 4435 | 150 | 3324 | 241 | <0.001 | 2.01% | 4.32% | 2.1 |
| Q61N | 29 | 4497 | 47 | 3427 | 76 | 0.002 | 0.64% | 1.35% | 2.1 |
| M46I* | 1129 | 3397 | 1819 | 1655 | 2948 | <0.001 | 24.94% | 52.36% | 2.1 |
| L10F** | 491 | 4035 | 786 | 2688 | 1277 | <0.001 | 10.85% | 22.63% | 2.1 |
| T74P | 76 | 4450 | 119 | 3355 | 195 | <0.001 | 1.68% | 3.43% | 2.0 |
| G73A** | 52 | 4474 | 80 | 3394 | 132 | <0.001 | 1.15% | 2.30% | 2.0 |
| A71V** | 1456 | 3070 | 2201 | 1273 | 3657 | <0.001 | 32.17% | 63.36% | 2.0 |
| L10I** | 1610 | 2916 | 2410 | 1064 | 4020 | <0.001 | 35.57% | 69.37% | 2.0 |
| H69K | 27 | 4499 | 40 | 3434 | 67 | 0.009 | 0.60% | 1.15% | 1.9 |
| A71I | 219 | 4307 | 322 | 3152 | 541 | <0.001 | 4.84% | 9.27% | 1.9 |
| M46L* | 451 | 4075 | 662 | 2812 | 1113 | <0.001 | 9.96% | 19.06% | 1.9 |
| K55N | 20 | 4506 | 29 | 3445 | 49 | 0.030 | 0.44% | 0.83% | 1.9 |
| M46V* | 34 | 4492 | 45 | 3429 | 79 | 0.016 | 0.75% | 1.30% | 1.7 |
| I72T | 271 | 4255 | 331 | 3143 | 602 | <0.001 | 5.99% | 9.53% | 1.6 |
| I50V | 119 | 4407 | 144 | 3330 | 263 | <0.001 | 2.63% | 4.15% | 1.6 |
| M36L** | 123 | 4403 | 145 | 3329 | 268 | <0.001 | 2.72% | 4.17% | 1.5 |
| I15V | 798 | 3728 | 927 | 2547 | 1725 | <0.001 | 17.63% | 26.68% | 1.5 |
| L89M | 107 | 4419 | 122 | 3352 | 229 | 0.003 | 2.36% | 3.51% | 1.5 |
| M36I** | 1536 | 2990 | 1698 | 1776 | 3234 | <0.001 | 33.94% | 48.88% | 1.4 |
| I62V | 1899 | 2627 | 2093 | 1381 | 3992 | <0.001 | 41.96% | 60.25% | 1.4 |
| N37D | 777 | 3749 | 826 | 2648 | 1603 | <0.001 | 17.17% | 23.78% | 1.4 |
| L90M* | 2460 | 2066 | 2491 | 983 | 4951 | <0.001 | 54.35% | 71.70% | 1.3 |
| G16E | 130 | 4396 | 131 | 3343 | 261 | 0.026 | 2.87% | 3.77% | 1.3 |
| K20M** | 239 | 4287 | 234 | 3240 | 473 | 0.006 | 5.28% | 6.74% | 1.3 |
| T74S | 360 | 4166 | 343 | 3131 | 703 | 0.003 | 7.95% | 9.87% | 1.2 |
| E35D | 1475 | 3051 | 1358 | 2116 | 2833 | <0.001 | 32.59% | 39.09% | 1.2 |
| R57K | 568 | 3958 | 517 | 2957 | 1085 | 0.003 | 12.55% | 14.88% | 1.2 |
| D60E | 581 | 3945 | 527 | 2947 | 1108 | 0.003 | 12.84% | 15.17% | 1.2 |
| L10V** | 428 | 4098 | 374 | 3100 | 802 | 0.055 | 9.46% | 10.77% | 1.1 |
| I93L | 1958 | 2568 | 1676 | 1798 | 3634 | <0.001 | 43.26% | 48.24% | 1.1 |
| L63P** | 3678 | 848 | 3105 | 369 | 6783 | <0.001 | 81.26% | 89.38% | 1.1 |
| Univariate analysis using a FC cut-off of 10-fold identified protease mutations with statistically significant associations with reduced | | | | | | | | | |
| # susceptibility to IDV/RTV. An odds ratio (OR) was calculated by dividing the proportion of samples with a given mutation that have FC >10 by | | | | | | | | | |
| # the proportion with the same mutation that have FC <10. OR >1 indicates positive association with reduced susceptibility while OR <1 indicates a | | | | | | | | | |
| # negative association. In the above table mutations are sorted by decreasing OR; only mutations with OR >1 are shown. | | | | | | | | | |

*Primary mutations in IDV interpretation algorithm
**Secondary mutations in IDV interpretation algorithm
†Secondary mutations added for IDV/RTV interpretation algorithm

Results indicated that protease mutations associated with greater than 10 FC included recognized primary mutations (M46I/L/V, G48M/S/V, V82A/F/S/T, I84A/V, and L90M) and both recognized and previously unrecognized secondary mutations (L10I/F/R/V, K20I/M/R/T, L24I, V32I, L33F, E34Q, M36I/L, K43T, I47A/V, I54A/L/M/S/T/V, L63P/S/A/T/Q/V/C, A71I/L/V/T, G73A/C/S/T, N88S/T, and L89V).

Application of exemplary rules and results for the identification of optimum numbers of mutation to be considered are presented in the following examples.

### 6.2 Example 2

### Discordance Rates for IDV Resistance Genotvping

In order to determine optimal rules a dataset (n=8551) was culled from the database described in Example 6.1 in May 2003. This dataset was filtered to exclude wildtype (genotypes with no known mutations associated to resistance to Protease Inhibitors) and redundant samples.

Genotype interpretation algorithms were developed using PERL scripts, convenient for parsing text files. The programs were run on desktop computers running Microsoft WINDOWS operating system.

An initial genotyping rule directed towards identifying IDV resistance defined as a phenotypic FC=2.5 was applied to the dataset. This phenotypic FC requirement classifies HIV samples with phenotypic FC of below 2.5 as being PS and those with a phenotypic FC of equal to or greater than 2.5 as being resistant. This rule identified genotypes as resistant (“GR”) if the sample contained one primary mutation among M46I/L/V, G48M/S/V, V82A/F/S/T, I84A/V, and L90M.

Results showed that 11.7% of the samples identified were incorrectly called (termed discordant samples) including 10% false negatives (GS-PR) and 1.7% false positives (GR-PS).

To decrease the number of discordant samples, secondary mutations (L10I/F/R/V, K20I/M/R/T, L24I, V32I, M36I/L/V, I54A/L/M/S/T/V, L63P/S/A/T/Q/V/C, A71I/L/V/T, G73A/C/S/T, and N88S/T) were added as additional condition, where a primary may count as a secondary mutation where it is not counted as a primary.

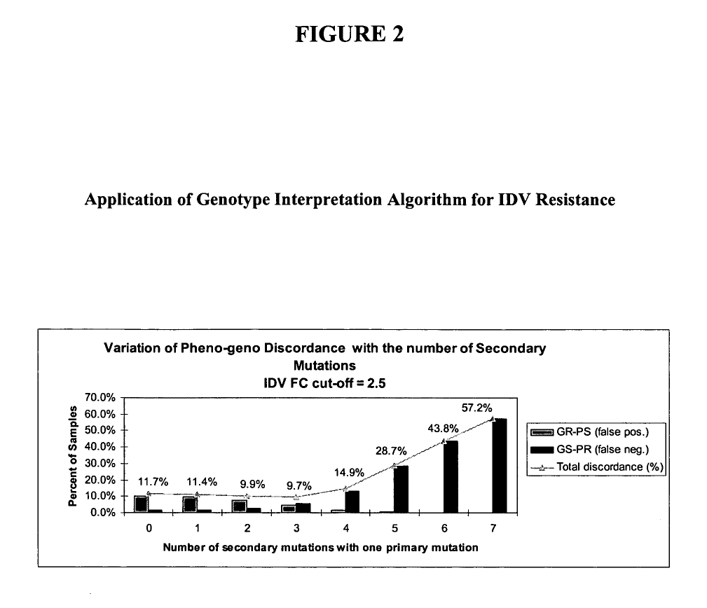

To select the best number of secondary mutations to minimize the number of discordant samples, eight genotyping rules were successively run, each with an increasing number of secondaries on the same dataset. Thus, the algorithm defined samples as GR if their genotype contained one primary mutation and a number of secondaries varying from 0 to 7. Exemplary results are shown in FIG. 2. Results demonstrated that the best number of secondary mutations associated with one primary mutation that minimizes the number of discordant samples (both false positives and false negatives) is 2 or 3.

### 6.3 Example 3

### Application of IDV Rule to IDV/RTV

Using the IDV algorithms applied in Example 2 above, it was ascertained if the same set of primaries and secondaries would have similar discordance levels and the same optimal number of secondaries when applied to IDV/RTV. In this case, a FC clinical cutoff of 10 was used to define samples as sensitive (samples with a FC less than 10) or resistant (samples with a FC equal to or greater than 10).

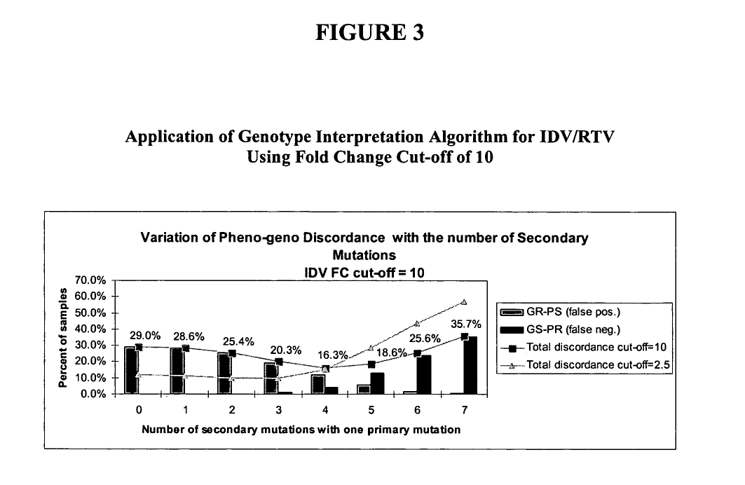

As shown in FIG. 3, the minimal discordance (16.3%) was found with the rule of selecting for genotypes with 1 primary mutation and at least 4 secondaries.

### 6.4 Example 4

### Identifying IDV/RTV Minimal Discordance

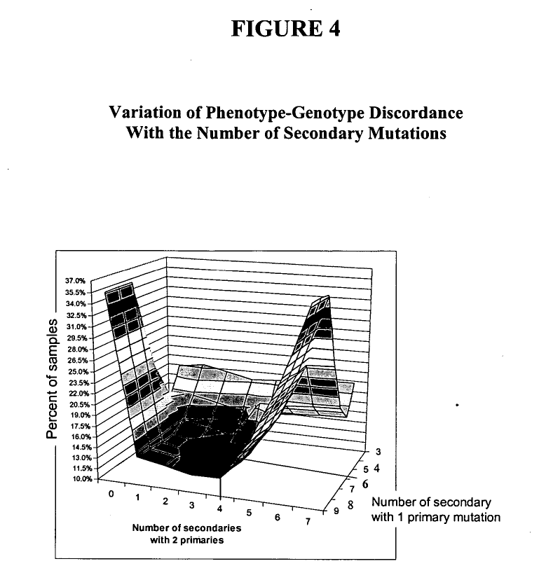

In order to reduce discordance levels associated with IDV/RTV genotype interpretation, the optimum set of primary and secondary mutations described in Example 1 was introduced into a new set of rules. As in Example 3, an FC cutoff of 10 was required for a sample to be PR. Exemplary results from the reiterative process of running different genotypic interpretation rules against the dataset are shown in FIG. 4.

FIG. 4 is a three-dimensional graph representing the percentage of discordant samples found with varying the number of secondary mutations from 0 to 7 associated with two primary mutations or varying number of secondary mutation from 3 to 9 associated with one primary mutation.

It was found that a minimum discordance of 12% is reached for the combined rules of selecting as GRs where the condition is met that one primary and at least six secondary mutations are identified OR two primaries and at least four secondary mutations are identified in the sample genotype.

### 6.5 Example 5

### Exemplary Decision Tree

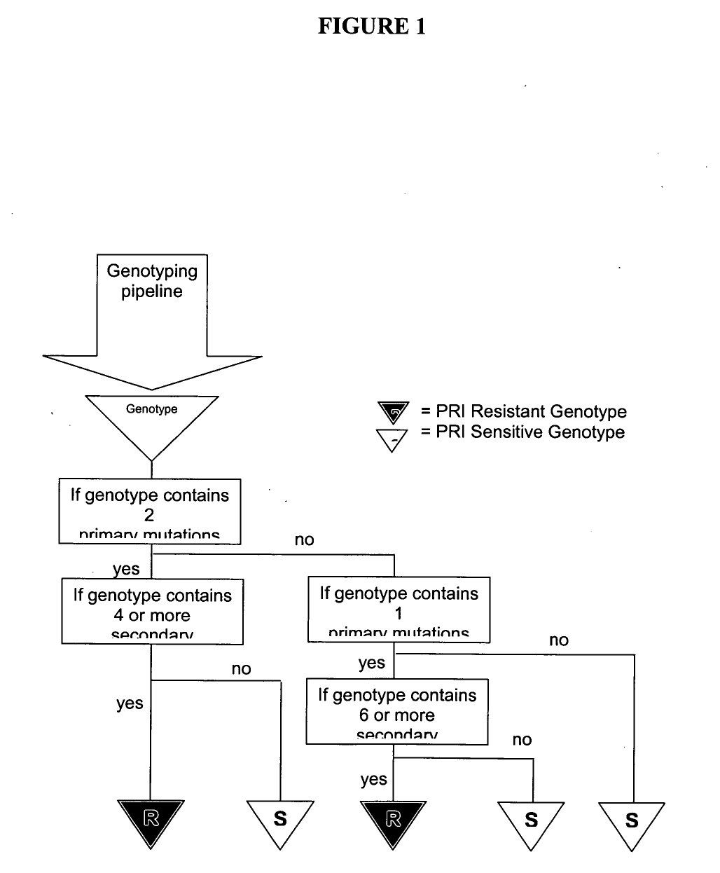

As explained in Example 4, a discordance level of 12% is reached in an algorithm that uses the following rule:
1 primary and 6 or more secondary mutations OR
2 primary and 4 or more secondary mutations
   to classify samples as GR, where the samples are defined as PR when having a 10 FC or greater. A decision tree is shown in FIG. 1 in order facilitate an understanding of the logic of how these conditions can be decided in an algorithm to classify genotypes. FIG. 1 is not intended to be a limitation or representation of the order of steps performed in a computer code performing any algorithm described herein.

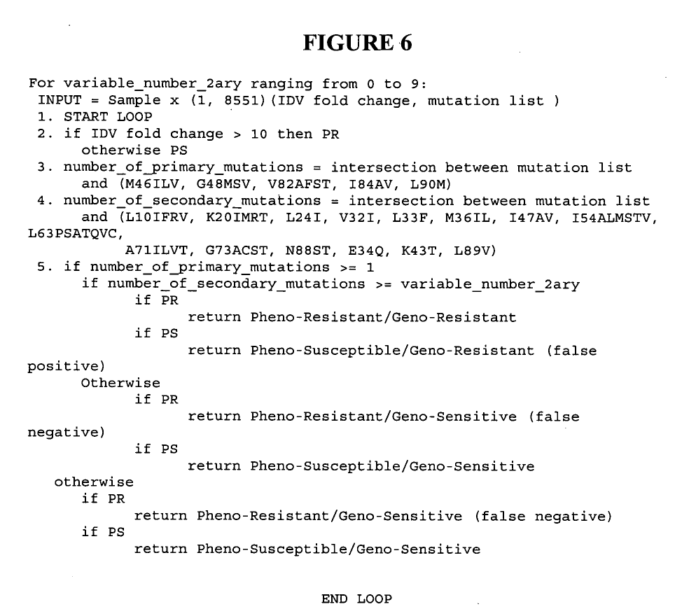

As another aid for facilitating an understanding how the steps in an algorithm could be implemented, FIG. 6 illustrates the key elements in an exemplary algorithm. For brevity, FIG. 6 is not intended to be a complete algorithm nor be syntactically correct in any programming language. It is intended merely to provide an example of the sorts ways the genotyping interpretation rules can be presented in an algorithm.

### 6.6 Example 6

### Genotype Interpretation Algorithm for IDV/RTV Resistance

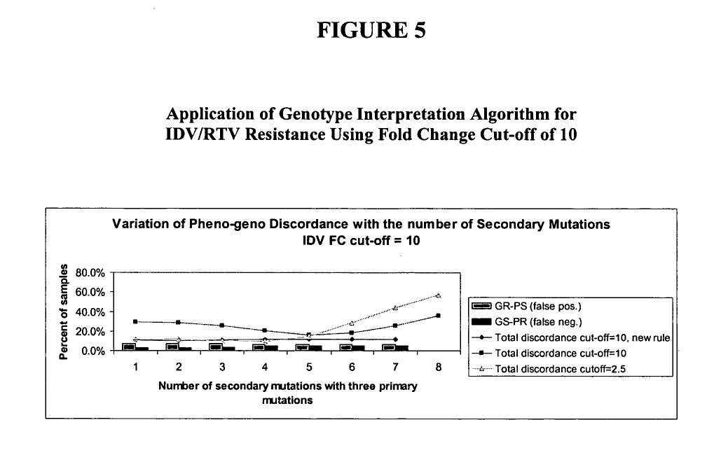

In order to further reduce discordance levels, additional rules were tested in conjunction with those identified in Example 4. FIG. 5 illustrates exemplary data obtained using the primary mutation set and secondary mutation set as identified in Example 1, setting PR to a 10 FC cutoff, and identifying discordance levels (combined false positives and false negatives) where different number of secondary mutations are determined in the presence of three primary mutations (primary mutations in the sample genotype in excess of three are counted as a secondary mutation).

Results indicate that a discordance level of 10.6% is reached using 10 FC cutoff to define a HIV sample as resistant, and an algorithm set up to identify genotypes meeting the condition of:
GR=one primary mutation and at least six secondary mutations OR
two primary mutations and at least four secondary mutations OR
three or more primary mutations and at least one secondary mutation
   (where primaries mutations are counted as secondary mutations if present and not counted as a primary mutation).

### 6.7 Example 7

### Confirming a Genotype Interpretation Algorithm with Two Naive Datasets

This example describes evaluation of the genotype interpretation rules for susceptibility to IDV/RTV described above. To this end, the discordance rate of the algorithm was calculated using a set of samples obtained subsequent to construction of the algorithm, and compared to the discordance rate reported in Example 6.

In addition, to confirm that the algorithm disclosed herein provides the best phenotypic prediction, we compared its performance with that of the interpretation rules for IDV published by two other groups, the Agence Nationale de Recherches sur le Sida (“ANRS”) (version 12; published December 2004; ANRS; Paris, France) and those by VGI (version 8, TruGene, Bayer Inc.; Berkeley, Cailf.).

The interpretation rules for genotypic susceptibility to IDV/RTV were tested on 2 datasets. Both datasets include a single sample per patient, and exclude samples wild-type (by genotypic criteria) for all Protease Inhibitors.

Dataset 1 includes all samples reported between 2000 and 2003. Dataset 1 was used to generate the genotype interpretation algorithm described in the examples above. This dataset contained 9228 individual samples. Dataset 2 includes all samples reported in 2004 and 2005, and was used as a naive dataset to assess the discordance rate of the genotype interpretation algorithm generated from Dataset 1. Dataset 2 contained 4634 individual samples.

The results of the analysis are summarized in Table 2, below.

TABLE 2

| | | number of | | | | | | | |
|---|---|---|---|---|---|---|---|---|---|
| | | samples | | | | | percent of | | |
| | | | GrPs | | | | samples | | |
| | | | excluding | | | | | | total |
| Algorithm | Dataset | GrPs | mixtures | GsPs | GrPr | GsPr | GrPs | GsPr | discordant |
| Disclosed | 1 | 1274 | 688 | 3996 | 3648 | 310 | 8.0% | 3.6% | 11.5% |
| Herein | | | | | | | | | |
| Disclosed | 2 | 593 | 349 | 1906 | 1985 | 150 | 7.9% | 3.4% | 11.4% |
| Herein | | | | | | | | | |
| ANRS* | 2 | 1627 | 1012 | 872 | 2120 | 15 | 25.2% | 0.4% | 25.6% |
| VGI* | 2 | 1018 | 639 | 1481 | 2060 | 75 | 15.0% | 1.8% | 16.8% |

*rules in use as of December 2004; not specifically intended for RTV-boosted IDV, but no specific rules for this regimen are available.

The discordance rate (11.4%) obtained for Dataset 2 for the algorithm disclosed above was the same as determined from Dataset 1, suggesting that the algorithm's accuracy is not an artifact of dataset optimization.

The algorithms published by ANRS and by VGI showed a discordance rate of 11.3% and 12.6%, respectively, on Dataset 1. The discordance rate was 25.6% and 16.8%, respectively, when calculated on Dataset 2. Thus, the algorithm provided herein better predicts IDV/r resistance for the new dataset than either the ANRS algorithm or the VGI algorithm.

The examples provided herein, both actual and prophetic, are merely embodiments of the present invention and are not intended to limit the invention in any way.

All publications and patent applications cited in this specification are herein incorporated by reference as if each individual publication or patent application were specifically and individually indicated to be incorporated by reference.

1. A method of determining whether a likelihood exists for reduced protease inhibitor (“PRI”) susceptibility of a Human Immunodeficiency Virus (“HIV”) population in a subject, comprising:
(a) identifying whether a nucleic acid obtained from HIV of the subject contains one or more primary mutations in the nucleic acid encoding codon 46, 48, 82, 84, or 90 of HIV protease;
(b) identifying whether the nucleic acid contains one or more secondary mutations in the nucleic acid encoding codon 10, 20, 24, 32, 33, 34, 36, 43, 46, 47, 48, 54, 63, 71, 73, 82, 84, 88, 89, or 90 of HIV protease; and
(c) determining whether a condition is met wherein
(i) the presence of one primary mutation and at least six secondary mutations are identified; or
(ii) the presence of two primary mutations and at least four secondary mutations are identified; or
(iii) three or more primary mutations and at least one secondary mutation are identified;
with the proviso that an identified primary mutation may not also be counted as a secondary mutation;
such that if it is determined that a condition in step (c) is met then the likelihood for reduced PRI susceptibility of the HIV population in the subject exists.

2. The method of claim 1, wherein the primary mutation encodes an amino acid in the HIV protease selected from the group consisting of M46I/L/V, G48M/S/V, V82A/F/S/T, I84A/V, and L90M and the secondary mutation encodes an amino acid in the HIV protease selected from the group consisting of L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M.

3. The method of claim 1, wherein the protease inhibitor is ritonavir-boosted indinavir.

4. The method of claim 3, wherein the HIV in the subject is about 10 times less susceptible to ritonavir-boosted indinavir than that of a reference HIV.

5. The method of claim 4, wherein the reference HIV is the NL4-3 strain of HIV.

6. The method of claim 1, wherein the PRI is IDV, the primary mutation encodes an amino acid in the HIV protease selected from the group consisting of M46I/L/V, G48M/S/V, V82A/F/S/T, I84A/V, and L90M, and the secondary mutation encodes an amino acid in the HIV protease selected from the group consisting of L10I/F/R/V, K20I/M/R/T, L241I, V32I, L33F, M36I/L, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, and L90M.

7. A method for assessing the effectiveness of ritonavir-boosted indinavir therapy in a HIV-infected subject comprising
determining whether a nucleic acid obtained from HIV of the subject contains
(i) one or more primary mutations where the one or more primary mutations are in the nucleic acid encoding codon 46, 48, 82, 84,or 90 of HIV protease, and
(ii) one or more secondary mutations where the one or more secondary mutations are in the nucleic acid encoding codon 10, 20, 24, 32, 33, 34, 36, 43, 46, 47, 48, 54, 63, 71, 73, 82, 84, 88, 89, or 90 of HIV protease,
in a combination of
one primary mutation and at least six secondary mutations, or
two primary mutations and at least four secondary mutations, or
three or more primary mutations and at least one secondary mutation,
wherein a mutation counted as a primary mutation may not also be counted as a secondary mutation,
such that the presence of such a combination indicates a decrease in susceptibility to ritonavir-boosted indinavir, thereby assessing the effectiveness of ritonavir-boosted indinavir therapy in the subject.

8. The method of claim 7, wherein the one or more primary mutations encode for an amino acid selected from the group consisting of M46/I/L/V, G48M/S/V, V82A/F/S/T, I84A/V, and L90M, and the one or more secondary mutations encode for an amino acid selected from the group consisting of L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M.

9. The method of claim 8, wherein the decrease in susceptibility to ritonavir-boosted indinavir therapy is about equal to or greater than a clinical cutoff value of 10-fold.

10. A computer implemented method of identifying a HIV population as being less susceptible to ritonavir-boosted indinavir therapy in a subject infected with the HIV population, comprising:
(a) inputting to a computer system data representing the genotype of the a nucleic acid encoding HIV protease obtained from HIV of the subject;
(b) performing a first comparison of the genotype of the nucleic acid encoding codons 46, 48, 82, 84, or 90 of HIV protease to a database in the computer wherein the database includes nucleic acid genotypes encoding mutant codons L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M, such that if a match is identified in the first comparison, a second comparison of the genotype of the nucleic acid encoding codons 10, 20, 24, 32, 33, 34, 36, 43, 46, 47, 48, 54, 63, 71, 73, 82, 84, 88, 89, or 90 of HIV protease to the database is performed; and
(c) determining whether a condition is met that
(i) one match is made in the first comparison and at least six matches are made in the second comparison; or
(ii) two matches are made in the first comparison and at least four matches are made in the second comparison; or
(iii) three or more matches are made in the first comparison and at least one match is made in the second comparison;
with the proviso that a match made in the first comparison may not be counted as a match in the second comparison;
such that the HIV population is identified as being less susceptible to ritonavir-boosted indinavir therapy in a subject infected with the HIV population if it is determined that a condition in step (c) is met.

11. The computer implemented method of claim 10, further comprising displaying a result indicating whether or not that the HIV population is identified as being less susceptible to ritonavir-boosted indinavir therapy in a subject infected with the HIV.

12. The method of claim 11, wherein the result is displayed on a tangible medium.

13. The method of claim 12, wherein the result is displayed on paper.

14. The method of claim 11, wherein the result is displayed on a computer screen.

15. A paper display of the result produced by the method of claim 12.

16. An article of manufacture that comprises computer-readable instructions for performing the method of claim 10.

17. The computer implemented method of claim 10, wherein the inputted data have been converted from a hybridization pattern of the HIV nucleic acid onto an oligonucleotide probe array attached to a solid phase.

18. A computer system that is configured to perform the method of claim 10.

19. A computer program product that identifies a HIV population as being less susceptible to ritonivir-boosted indinavir drug treatment in a subject infected with HIV, comprising:
(a) a computer code that receives input corresponding to the genotype of the HIV nucleic acid encoding HIV protease obtained from the subject;
(b) a computer code that performs a first comparison to determine if an amino acid encoded by HIV protease codons 46, 48, 82, 84 and 90 of the HIV nucleic acid matches one or more of mutant amino acids M46I/L/V, G48M/S/V, V82A/F/S/T, I84A/V, and L90M of HIV protease;
(c) a computer code that performs a second comparison to determine if an amino acid encoded by HIV protease codons 10, 20, 24, 32, 33, 34, 36, 43, 46, 47, 48, 54, 63, 71, 73, 82, 84, 88, 89 and 90 of the HIV nucleic acid matches one or more of mutant amino acids L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, and L90M;
(d) a computer code that determines whether a condition is met that
(i) one match is made in the first comparison and at least six matches are made in the second comparison, or
(ii) two matches are made in the first comparison and at least four matches are made in the second comparison, or
(iii) three or more matches are made in the first comparison and at least one match is made in the second comparison,
with the proviso that a match made in the first comparison may not be counted as a match in the second comparison,
wherein the HIV population is identified as being less susceptible to ritonivir-boosted indinavir drug treatment in a subject infected with HIV if such a condition is determined to be met;
(e) a computer code that conveys a result representing whether or not the HIV is identified as being less susceptible to ritonivir-boosted indinavir drug treatment in a subject infected with HIV to an output device; and
(f) a computer readable medium that stores the computer codes.

20. The computer program product of claim 18, wherein the input has been obtained from a hybridization pattern of the HIV nucleic acid onto an oligonucleotide array attached to a solid phase.

21. A tangible medium storing the result conveyed to the output device in claim 19.

22. The tangible medium of claim 21 that is a printout.

23. The tangible medium of claim 21 that is a CD or DVD.

24. A system of providing information of whether a HIV-infected subject is resistant to ritonavir-boosted indinavir comprising:
(a) obtaining a genotype for HIV protease obtained from the subject;
(b) identifying the presence or absence of a primary mutation in the HIV comprising M46I/L/V, G48M/S/V, V82A/F/S/T, I84A/V, or L90M of HIV protease;
(c) identifying the presence or absence of a secondary mutation in the HIV comprising L10I/F/R/V, K20I/M/R/T, L24I, V32I, L33F, E34Q, M36I/L, K43T, M46I/L/V, I47A/V, G48M/S/V, I54A/L/M/S/T/V, L63P/S/A/T/Q/V/C, A71I/L/V/T, G73A/C/S/T, V82A/F/S/T, I84A/V, N88S/T, L89V, or L90M of HIV protease;
(d) determining whether a condition is met wherein:
(i) the presence of one primary mutation and at least six secondary mutations are identified, or
(ii) the presence of two primary mutations and at least four secondary mutations are identified, or
(iii) three or more primary mutations and at least one secondary mutation are identified,
wherein a primary mutation counted as a secondary mutation may not also be counted as a secondary mutation;
such that if the condition (i), (ii) or (iii) is met, then the subject is resistant to ritonavir-boosted indinavir; and
(e) preparing a tangible medium comprising an indication of whether or not the subject is resistant to ritonavir-boosted indinavir as determined in step (d).

25. The system of claim 24, further comprising conveying the tangible medium to the subject or a health care provider.
